SEQUENCE LISTING (39 sequences)

SEQ ID NO: 1 - DNA, 23 bp, Artificial
  synthetic oligonucleotide
ccactattcaagtacccagtcgg

SEQ ID NO: 2 - protein, 7 aa, Artificial
  synthetic polypeptide
HYSSTQS

SEQ ID NO: 3 - DNA, 23 bp, Artificial
  synthetic oligonucleotide
ccactatttaagtacccagtcgg

SEQ ID NO: 4 - protein, 7 aa, Artificial
  synthetic polypeptide
HYLSTQS

SEQ ID NO: 5 - DNA, 7548 bp, Artificial
  synthetic plasmid construct
cacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggcccaaatacctcgagggagcaaggcaggtggtcgacggtatcgataagcttgatatcgaattctaactgtctccaccgataatgttgtataatacccgtgaaatcatagcacatgatatatcatcacccggaggccggttattttcggcggcggcaaaaatatttggtataattatggaaatacaaaaaggggaaccattaaaggttgaggaggggattgataagagaatctaataattgtaaagttgagaaaatcataataaaaataattactagagaccccgggatgggtgacggtgctggtttaattaacatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggatccatggataattgctccggttctcgtaggagggaccgtctacatgttaagctgaaatctttaaggaataagatacacaagcaactacacccaaattgtcgttttgatgatgctacgaagacaagttaagcggccgccaccgcggtggagctctaagcaaatagctaaattatatacgaattaatattatgattaagtgtttacgtgagtgcgatatttttattactatcttatacagttgtatatactctataaaatgagttgtctattaattaacgcgatgaatgctttctgggtttacctctccaacaactctagtttacttctcaatacattcaattgtatttgatttgtcaatacttcatcattaatcaattctatagttttgtttttctcgtttatttccaaatttaatgcatcaattttattattcaatttgtcgttgattttggttaatgattttatggtttgatctctggcattgattgtttgtgttagtttttcattattgataattaaattatttaagttagttatcaactcggtgttttcaagtttcaagttttcaatttctttagagtttattagatttgtcaaagtttctgaattgcttgattggtcctgtagaagagtatttgttgttgtggataattgattcaatttttgagacaattgctggaaggcgttgaaatatctagcatcaatctcatggtttttttcccgagagtctcgtagattcaattgttttaatatatcttgggaccactcttgatttgaactcatggaaattaaactgggtgttgtgttgtggtgtaatgattgtaccccctttgcttataattgtgtggcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggttattactgagtagtatttatttaagtattgtttgtgcacttgccgatctatgcggtgtgaaataccg

SEQ ID NO: 6 - DNA, 1865 bp, Homo sapiens
agcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaataatggtcttttttttttttttaatgtttcttcctctgtttggctttgccattgtgcgatttggaaaaaccacttggaagaagggactttcctgcaaaaccttaaagactggttaaattacagggcctaggaagtcagtggagccccttgactgacaaagcttagaaaggaactgaaattgcttctttgaatatggattttaggcggggcgtggtggctcacgcctataatcccagcacgttgggaggccaacgcgggtggatcacctgaggtcaggagttcgagaccagcctgactaacatggtgaaaccctgtctctactaaaaatacaaaattagtcaggcgtggtggtgcacacctgtaatcccagctacttgggagactgaggcaggaggatcgcttgaacccgggaggcagaggttgcggtgagccgagatcatgccattgcactccagcctgggcaacagagcaagactctgtgtcaaaaaaaaaaaaagaatatagatttttaaatggc

SEQ ID NO: 7 - DNA, 2889 bp, Homo sapiens
gttttctgtctagctccgaccggctgaggcggcgcggcagcggagggacggcagtctcgcgcggctactgcagcactggggtgtcagttgttggtccgacccagaacgcttcagttctgctctgcaaggatatataataactgattggtgtgcccgtttaataaaagaatatggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagggcctgcagttgccatcacccactgcaaccagccagctcccgctcgaatctgatgctgtggaatgcttaaattaccaacactataaaggaagtgactttgactgcgagttgaggctgttgattcatcagagtctagcaggaggaattattggggtcaaaggtgctaaaatcaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtatgcagatgttgaaggattctaatgcaagatattttttcttttttatagtgtgaagcagtattctggaaagtttttctaagactagtgaagaactgaaggagtcctgcatctttttttttttatctgcttctgtttaaaaagccaacattcctctgcttcataggtgttctgcatttgaggtgtagtgaaatctttgctgttcaccagatgtaatgttttagttccttacaaacagggttgggggggggaagggcgtgcaaaaactaacattgaaattttgaaacagcagcagagtgagtggattttatttttcgttattgttggtggtttaaaaaattccccccatgtaattattgtgaacaccttgctttgtggtcactgtaacatttggggggtgggacagggaggaaaagtaacaatagtccacatgtccctggcatctgttcagagcagtgtgcagaatgtaatgctcttttgtaagaaacgttttatgatttttaaaataaatttagtgaacctatttttggtggtcattttttttttaagacagtcattttaaaatggtggctgaatttcccaacccacccccaaactaaacactaagtttaattttcagctcctctgttggacatataagtgcatctcttgttggacataggcaaaataacttggcaaacttagttctggtgatttcttgatggtttggaagtctattgctgggaagaaattccatcatacatattcatgcttataataagctggggattttttgtttgtttttgcaaatgcttgcccctacttttcaacaattttctatgttagttgtgaagaactaaggtggggagcagtactacaagttgagtaatggtatgagtatataccagaattctgattggcagcaagttttattaatcagaataacacttggttatggaagtgactaatgctgaaaaaattgattatttttattagataatttctcacctatagacttaaactgtcaatttgctctagtgtcttattagttaaactttgtaaaatatatatatacttgtttttccattgtatgcaaattgaaagaaaaagatgtaccatttctctgttgtatgttggattatgtaggaaatgtttgtgtacaattcaaaaaaaaaaaagatgaaaaaagttcctgtggatgttttgtgtagtatcttggcatttgtattgatagttaaaattcacttccaaataaataaaacacccatgatgctagatttgatgtgtgcccgatttgaacaagggttgattgacacctgtaaaatttgttgaaacgttcctcttaaaaggaaatatagtaatcttatgta

SEQ ID NO: 8 - DNA, 2614 bp, Artificial
  synthetic construct
gtacaaaaaagcaggctccaccatggatgtgcgccgtctgaaggtgaacgaacttcgcgaggagctgcagcgccgcggcctggacactcgaggcctcaaggccgagcttgctgagcggctgcaggcggcgttggaggccgaggagcctgacgacgagcgggagctcgacgccgacgacgaaccggggcgacccgggcacatcaacgaggaggtcgagaccgaggggggctccgagctggaggggaccgcgcagccaccgccgcccgggctgcagccgcacgcggagcccggctgctactcggggccggacggacattatgccatggacaatattaccaggcagaaccaattctacgatacccaagtcatcaaacaagaaaacgagtcaggctacgagaggagaccactggaaatggagcagcagcaggcctatcgtccagaaatgaagacagagatgaagcaaggagcacccaccagcttcctcccgcctgaagcttctcaactcaagccagacaggcagcaattccagagtcgaaagaggccttatgaagaaaaccggggacgggggtactttgagcaccgagaggataggaggggccgctctcctcagcctcctgctgaagaggatgaagatgactttgatgatacccttgttgctattgacacctataactgcgacctccacttcaaggtggcccgagatcggagtagtggctatccgctcacaattgagggctttgcatacctgtggtcaggagcccgtgccagctatggggtcagaaggggccgtgtatgcttcgagatgaagatcaatgaggaaatctccgtgaagcaccttccgtctacagagcctgacccccacgtggtccgtatcggctggtccctggactcctgcagcacccagctaggcgaagagcctttctcctatggctatggaggcactgggaagaagtccaccaatagccggtttgaaaactacggagacaagtttgcagagaacgatgtgattggctgctttgcggattttgaatgtggaaatgacgtggaactgtcttttaccaagaatggaaagtggatgggcattgctttccgaatccagaaggaagccttggggggtcaggccctctatcctcatgtcctggtgaagaattgcgcagtggagttcaacttcggacagagagcagagccctactgttctgtcctcccggggtttaccttcatccagcaccttccccttagtgagcgtatccggggcaccgttggaccaaagagcaaggcagaatgtgagattctgatgatggtgggcctgcctgctgctggcaagaccacatgggccatcaaacatgcagcctccaacccttccaagaagtacaacatcctgggtaccaatgccatcatggataagatgcgggtgatgggcctacgccggcagcggaactatgctggccgctgggatgtcctgatccagcaggccacccagtgcctcaaccgcctcatccagattgctgcccgcaagaaacgcaactatatcctagatcagacaaatgtttatgggtcagcccagagacgaaaaatgagaccatttgaaggcttccagcgcaaagctattgtaatttgtcccactgacgaggacctaaaagaccgaacaataaagcgaaccgacgaggaagggaaggatgtcccagatcatgcggtcttagaaatgaaagccaacttcacgttgccagatgttggggacttcctggatgaggttctgttcattgagctgcagcgggaggaagcggacaagctagtgaggcagtacaacgaggaaggccgcaaggctgggccaccccctgaaaagcgctttgacaaccgaggtggtggtggcttccggggccgcgggggtggtggtggcttccagcgctatgaaaaccgaggaccccctggaggcaaccgtggcggcttccagaaccgagggggaggcagcggtggaggaggcaactaccgaggaggtttcaaccgcagcggaggtggtggctatagccagaaccgctggggtaacaacaaccgggataacaacaactccaacaacagaggcagctacaaccgggctccccagcaacagccgccaccacagcagcctccgccaccacagccaccaccccagcagccaccgccaccacccagctacagccctgctcggaaccccccaggggccagcacctacaataagaacagcaacatccctggctcaagcgccaataccagcacccccaccgtcagcagctacagccctccacagccgagttacagccagccaccctacaaccagggaggttacagccagggctacacagccccaccgcctccacctccaccaccacctgcctacaactatgggagctacggcggttacaacccggccccctataccccaccgccaccccccaccgcacagacctaccctcagcccagctataaccagtatcagcagtatgcccagcagtggaaccagtactatcagaaccagggccagtggccgccatactacgggaactacgactacgggagctactccgggaacacacagggtggcacaagtacacagtgaatccacccagctttcttgtac

SEQ ID NO: 9 - DNA, 1444 bp, Homo sapiens
ggagaaggggtggggcagggtatcgctgactcagcagcttccaggttgctctgatgatatattaaggctcctgaatcctaagagaatgttggtgaagatcttaacaccacgccttgagcaagtcgcaagagcgggaggacacagaccaggaaccgagaagggacaagcacatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacgccagcaaagcagtatgctcccgatcaagtagatttttaaaaaatcagagtgggccgggcgcggtggctcacgcctgtaatcccagcactttggaggccaaggcgggtggatcacgaggtcaggagatcgagaccatcctggctaacacggtgaaaccctgtctctactaaaaatacaaaaaattagccaggcgtggtggcgggcgcctgtagtcccagctactctggaggctgaggcaggagagtagcgtgaacccgggaggcagagcttgcggtgagccgagattgcgctactgcactccagcctgggcgacagtaccagactccatctcaaaaaaaaaaaaaccagactgaattaattttaactgaaaatttctcttatgttccaagtacacaatagtaagattatgctcaatattctcagaataattttcaatgtattaatgaaatgaaatgataatttggcttcatatctagactaacacaaaattaagaatcttccataattgcttttgctcagtaactgtgtcatgaattgcaagagtttccacaaacact

SEQ ID NO: 10 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 11 - DNA, 1560 bp, Homo sapiens
cacagagcttcaaaaaaagagcgggacagggacaagcgtatctaagaggctgaacatgaatccacagatcagaaatccgatggagcggatgtatcgagacacattctacgacaactttgaaaacgaacccatcctctatggtcggagctacacttggctgtgctatgaagtgaaaataaagaggggccgctcaaatctcctttgggacacaggggtctttcgaggccaggtgtatttcaagcctcagtaccacgcagaaatgtgcttcctctcttggttctgtggcaaccagctgcctgcttacaagtgtttccagatcacctggtttgtatcctggaccccctgcccggactgtgtggcgaagctggccgaattcctgtctgagcaccccaatgtcaccctgaccatctctgccgcccgcctctactactactgggaaagagattaccgaagggcgctctgcaggctgagtcaggcaggagcccgcgtgaagatcatggactatgaagaatttgcatactgctgggaaaactttgtgtacaatgaaggtcagcaattcatgccttggtacaaattcgatgaaaattatgcattcctgcaccgcacgctaaaggagattctcagatacctgatggatccagacacattcactttcaactttaataatgaccctttggtccttcgacggcgccagacctacttgtgctatgaggtggagcgcctggacaatggcacctgggtcctgatggaccagcacatgggctttctatgcaacgaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgcatcttcgctgcccgcatctatgattacgaccccctatataaggaggcgctgcaaatgctgcgggatgctggggcccaagtctccatcatgacctacgatgagtttgagtactgctgggacacctttgtgtaccgccagggatgtcccttccagccctgggatggactagaggagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacaccagcaaagcaatgtgctcctgatcaagtagattttttaaaaatcagagtcaattaattttaattgaaaatttctcttatgttccaagtgtacaagagtaagattatgctcaatattcccagaatagttttcaatgtattaatgaagtgattaattggctccatatttagactaataaaacattaagaatcttccataattgtttccacaaacactaaaaaaaaaaaaaaaaaaaaaaa

SEQ ID NO: 12 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 13 - DNA, 4104 bp, Streptococcus pyogenes
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttggcagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaatctacaatcaattatttgaagaaaaccctattaacgcaagtagagtagatgctaaagcgattctttctgcacgattgagtaaatcaagacgattagaaaatctcattgctcagctccccggtgagaagagaaatggcttgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcgcctaccatgatttgctaaaaattattaaagataaagattttttggataatgaagaaaatgaagatatcttagaggatattgttttaacattgaccttatttgaagatagggggatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaggtgtctggacaaggccatagtttacatgaacagattgctaacttagctggcagtcctgctattaaaaaaggtattttacagactgtaaaaattgttgatgaactggtcaaagtaatggggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctacaaaatggaagagacatgtatgtggaccaagaattagatattaatcgtttaagtgattatgatgtcgatcacattgttccacaaagtttcattaaagacgattcaatagacaataaggtactaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 14 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEIAKVDDSFFHRLEESFLVEEDKKHERHPIFGNIVDEVAYHEKYPTIYHLRKKLADSTDKADLRLIYLALAHMIKFRGHFLIEGGLNPDNSDVDKLFIQLVQTYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDLQKAQVSGQGDSLHEHIANLAGSPAIKKGILQTVKVVDELVKVMGRHKPENIVIEMARENQTTQKGQKNSRERMKRIEEGIKELGSDILKEYPVENTQLQNEKLYLYYLQNGRDMYVDQELDINRLSDYDVDHIVPQSFLKDDSIDNKVLTRSDKNRGKSDNVPSEEVVKKMKNYWKQLLNAKLITQRKFDNLTKAERGGLSELDKAGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDIRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELVGITIMERSSFEKDPVDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIGRNRYKSIKEVLDATLIHQSITGLYETRIDLSQLGGD

SEQ ID NO: 15 - protein, 1368 aa, Artificial
  synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 16 - protein, 1368 aa, Artificial
  synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 17 - protein, 1368 aa, Artificial
  synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 18 - protein, 5 aa, Artificial
  synthetic polypeptide
GGGGS

SEQ ID NO: 19 - protein, 5 aa, Artificial
  synthetic polypeptide
EAAAK

SEQ ID NO: 20 - protein, 16 aa, Artificial
  synthetic polypeptide
SGSETPGTSESATPES

SEQ ID NO: 21 - protein, 9 aa, Artificial
  synthetic polypeptide
LAGLIDADG

SEQ ID NO: 22 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 23 - protein, 198 aa, Homo sapiens [sequence omitted]

SEQ ID NO: 24 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 25 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 26 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 27 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 28 - DNA, 45 bp, Artificial
  synthetic oligonucleotide
ctgctgcccgacaaccactattcaagtacccagtcggccctgagc

SEQ ID NO: 29 - DNA, 45 bp, Artificial
  synthetic oligonucleotide
ctgctgcccgacaaccactatttaagtacccagtcggccctgagc

SEQ ID NO: 30 - DNA, 45 bp, Artificial
  synthetic oligonucleotide
ctgctgcccgacaacyaytatttaagtacccagtcggccctgagc

SEQ ID NO: 31 - DNA, 35 bp, Homo sapiens
gcgcacctcatggaatcccttctgcagcacctgga

SEQ ID NO: 32 - DNA, 35 bp, Artificial
  synthetic oligonucleotide
gcgcacctcatggaatbyyttctgcagcacctgga

SEQ ID NO: 33 - DNA, 35 bp, Artificial
  synthetic oligonucleotide
gcgcacctyatggaatbyyttctgcagcacctgga

SEQ ID NO: 34 - DNA, 45 bp, Homo sapiens
ctggaggaggaagggcctgagtccgagcagaagaagaagggctcc

SEQ ID NO: 35 - DNA, 45 bp, Artificial
  synthetic oligonucleotide
ctggaggaggaagggcctgagtyygagcagaagaagaagggctcc

SEQ ID NO: 36 - DNA, 45 bp, Artificial
  synthetic oligonucleotide
ctggaggaggaagggcctgagtbygagcagaagaagaagggctcc

SEQ ID NO: 37 - DNA, 35 bp, Artificial
  synthetic oligonucleotide
gcgcacctyatggaatyyyttctgcagcacctgga

SEQ ID NO: 38 - DNA, 45 bp, Artificial
  synthetic oligonucleotide
ctggaggaggaagggcctgagtycgagcagaagaagaagggctcc

SEQ ID NO: 39 - DNA, 45 bp, Artificial; identical to SEQ ID NO: 36
  synthetic oligonucleotide

(19) United States
(12) Patent Application Publication
(10) Pub. No.: US 2021/0108188 A1
(43) Pub. Date: Apr. 15, 2021

(54) NON-COVALENT SYSTEMS AND METHODS FOR DNA EDITING

(71) Applicant: Regents of the University of Minnesota, Minneapolis, MN (US)

(72) Inventors: Reuben S. Harris, St. Paul, MN (US); Jennifer McCann, Savage, MN (US); Daniel James Salamango, Crystal, MN (US)

(21) Appl. No.: 17/067,401

(22) Filed: Oct. 9, 2020

Related U.S. Application Data
(60) Provisional application No. 62/913,435, filed on Oct. 10, 2019.

(51) Int. Cl. C12N 9/78; C12N 9/22; C12N 15/113; C12N 15/85

(52) U.S. Cl. CPC C12N 9/78; C12N 9/22; C12N 15/113; C12N 2800/80; C12Y 305/04; C12N 2310/20; C12N 15/85

(57) ABSTRACT

This document relates to materials and methods for DNA base editing with reduced off-target mutations. In particular, this document relates to materials and methods that include using fusion proteins containing a Cas9 molecule and an APOBEC-interacting molecule to achieve specific DNA edits with reduced levels of off-target edits.

### CROSS-REFERENCE TO RELATED APPLICATIONS

This application claims priority from U.S. Provisional Application Ser. No. 62/913,435, filed Oct. 10, 2019. The disclosure of the prior application is considered part of (and is incorporated by reference in) the disclosure of this application.

### STATEMENT AS TO FEDERALLY SPONSORED RESEARCH

This invention was made with government support under CA234228 awarded by the National Institutes of Health. The government has certain rights in the invention.

### TECHNICAL FIELD

This document relates to materials and methods for DNA base editing with reduced off-target mutations. In particular, this document relates to materials and methods that include using fusion proteins containing a Cas9 molecule and an APOBEC-interacting molecule to achieve specific DNA edits with reduced levels of off-target edits.

### BACKGROUND

Cytosine base editors (CBEs) typically include an apolipoprotein B mRNA editing enzyme, catalytic polypeptide-like (APOBEC) deaminase (e.g., rat APOBEC1) fused covalently to the N-terminal end of a Cas9 nickase [e.g., Cas9n (D10A); see, e.g., FIG. 1A and Komor et al., Nature 533, 420-424, 2016]. Appropriate guide (g)RNAs are able to target this assembly to specific genomic cytosine bases and facilitate high frequency editing. In fact, editing efficiencies of 10% to 90% can be achieved, depending on variables such as the distance between target cytosine and the protospacer adjacent motif (PAM) (Gaudelli et al., Nature 551, 464-471, 2017; and Komor et al., supra)—a two to six base pair DNA sequence immediately following the DNA sequence targeted by Cas9, without which Cas9 will not bind DNA. This technology is prone to a number of off-target effects, however, including RNA editing (Grunewald et al., Nature 569, 433-437, 2019; and Zhou et al., Nature 571, 275-278, 2019), random genomic DNA editing (Kim et al., Nat Biotechnol 35, 475-480, 2017; Gehrke et al., Nat Biotechnol 36, 977-982, 2018; Zuo et al., Science 364, 289-292, 2019; and Jin et al., Science 364, 292-295, 2019), and most frequently, target-adjacent editing (Gaudelli et al., supra; Komor et al., supra; Kim et al., supra; Coelho et al., BMC Biol 16, 150, 2018; and Kim et al., Nat Biotechnol 35, 371-376, 2017). The latter problem is due to deamination of single-stranded (ss)DNA cytosines located adjacent to the desired target cytosine in the same gRNA-displaced R-loop (a single-stranded DNA substrate that can be attacked by an APOBEC enzyme), as depicted in FIG. 1A. This issue has been diminished—but not eliminated—by mutating APOBEC1 (Grunewald et al., supra; Zhou et al., supra; Kim et al., Nat Biotechnol 35, 371-376, 2017; and Koblan et al., Nat Biotechnol 36, 843-846, 2018), replacing APOBEC1 with different DNA deaminase family members (St. Martin et al., Nucleic Acids Res 46, e84, 2018; St. Martin et al., Scientific Reports 9, 497, 2019; Zong et al., Nat Biotechnol 36, 950-953, 2018; Wang et al., Nat Biotechnol 36, 946-949, 2018; Komor et al., Sci Adv 3, eaao4774, 2017; Ma et al., Nat Methods 13, 1029-1035, 2016; and Hess et al., Nat Methods 13, 1036-1042, 2016), mutating Cas9 (Kim et al., Nat Biotechnol 35, 371-376, 2017; Hu et al., Nature 556, 57-63, 2018; Thuronyi et al., Nat Biotechnol, 2019; Huang et al., Nat Biotechnol 37, 820, 2019; Rees et al., Nat Commun 8, 15790, 2017; Endo et al., Nat Plants 5, 14-17, 2019; and Li et al., Nat Biotechnol 36, 324-327, 2018), and using different Cas enzymes/complexes (Koblan et al., supra; Komor et al. 2017, supra; Li et al., supra; and Kleinstiver et al., Nat Biotechnol 37, 276-282, 2019).

### SUMMARY

This document is based, at least in part, on the discovery of methods for using non-covalent methods to “attract” a DNA cytosine deaminase to a particular genomic cytosine target. The materials and methods provided herein can decouple the fates of on-target and target-adjacent editing events, thus enhancing the likelihood that a precise, single base substitution mutation will be obtained in the absence of any adjacent editing events. As described herein, a key to implementing this non-covalent strategy is using cytosine deaminase-interacting polypeptides (also referred to herein as APOBEC-interacting polypeptides) that can bind the deaminase without blocking access to the active site. Such interacting proteins can be tethered to a Cas9n polypeptide and used to “attract” a cytosine deaminase (e.g., an APOBEC enzyme, including exogenous and endogenous APOBEC enzymes) to edit a particular genomic target cytosine. The system described herein is referred to as “MagnEdit,” and is illustrated in FIG. 1B.

In a first aspect, this document features a fusion polypeptide containing (a) an APOBEC-interacting polypeptide, and (b) a Cas9 polypeptide. The APOBEC-interacting polypeptide can be N-terminal of the Cas9 polypeptide. The APOBEC-interacting polypeptide can be a heterogeneous nuclear ribonucleoprotein U-like (hnRNPUL1) polypeptide. The hnRNPUL1 polypeptide can be encoded by a nucleic acid sequence containing the sequence set forth in SEQ ID NO:8, or a sequence having at least about 90% identity to SEQ ID NO:8. The APOBEC-interacting polypeptide can be an antibody or an antigen binding portion thereof. The antibody or antigen-binding portion thereof can be a single chain antibody or an antigen binding portion thereof. The Cas9 polypeptide can be encoded by a nucleic acid sequence containing the sequence set forth in SEQ ID NO:13, or a sequence having at least about 90% identity to SEQ ID NO:13, with the proviso that in the encoded Cas9 polypeptide, that the amino acid at the position corresponding to position 10 of SEQ ID NO:14 is A1a, the amino acid at the position corresponding to position 840 of SEQ ID NO:14 is A1a, or the amino acids at the positions corresponding to positions 10 and 840 of SEQ ID NO:14 are A1a.

In another aspect, this document features a nucleic acid molecule containing a nucleotide sequence encoding a fusion polypeptide provided herein. The nucleic acid molecule can be an expression vector.

In another aspect, this document features a host cell containing a nucleic acid molecule provided herein.

In yet another aspect, this document features a method for inducing DNA base editing at a specific DNA target in a cell, where the method includes introducing into the cell (a) a first nucleic acid encoding a fusion polypeptide, where the first nucleic acid includes (i) a sequence encoding an APOBEC-interacting polypeptide, and (ii) a sequence encoding a Cas9 polypeptide; and (b) a guide RNA (gRNA) targeted to the specific DNA target. The method can further include introducing into the cell (c) a nucleic acid encoding an APOBEC polypeptide. The APOBEC polypeptide can be an APOBEC3B polypeptide. The sequence encoding the APOBEC-interacting polypeptide can be 5′ of the sequence encoding the Cas9 nickase. The APOBEC-interacting polypeptide can be a hnRNPUL1 polypeptide. The hnRNPUL1 polypeptide can be encoded by a nucleic acid sequence containing the sequence set forth in SEQ ID NO:8, or a sequence having at least about 90% identity to SEQ ID NO:8. The Cas9 polypeptide can be encoded by a nucleic acid sequence containing the sequence set forth in SEQ ID NO:13, or a sequence having at least about 90% identity to SEQ ID NO:13, with the proviso that in the encoded Cas9 polypeptide, the amino acid at the position corresponding to position 10 of SEQ ID NO:14 is A1a, the amino acid at the position corresponding to position 840 of SEQ ID NO:14 is A1a, or the amino acids at the positions corresponding to positions 10 and 840 of SEQ ID NO:14 are A1a. The cell can be a primary human cell. The cell can be a stem cell, a lymphocyte, or a hepatocyte.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C illustrate covalent CBE technology versus non-covalent MagnEdit technology for DNA cytosine base editing. FIG. 1A is a schematic of current CBE methodology showing an APOBEC-Cas9n/gRNA editosome engaging the eGFP Leu202 reporter. Target-adjacent mutations are indicated by X's. FIG. 1B is a schematic of MagnEdit, showing an interactor-Cas9n/gRNA complex recruiting an untethered A3B to the eGFP Leu202 reporter. FIG. 1C is a graph plotting quantification of episomal eGFP reporter editing activity of the indicated MagnEdit complexes in 293T cells (n=3 biologically independent experiments, average±SD, p<0.0001 by unpaired student t-test for circled histogram bars). Immunoblots from a representative experiment are shown below the graph. The inset schematic shows the eGFP Leu202 reporter, the DNA region matching the gRNA, and the target cytosine. Unedited L202 reporter, SEQ ID NO:1; unedited eGFP sequence, SEQ ID NO:2; edited L202 reporter, SEQ ID NO:3; edited eGFP sequence, SEQ ID NO:4.

FIGS. 2A-2D show chromosomal DNA editing by MagnEdit. FIG. 2A is a graph plotting quantification of chromosomal eGFP reporter editing activity of the indicated MagnEdit complexes in 293T cells (n=3 biologically independent experiments, average±SD, p<0.0009 by unpaired student t-test for circled histogram bars). Immunoblots from a representative experiment are shown below the graph. FIGS. 2B-2D are graphs plotting chromosomal eGFP editing activity for reactions containing the indicated components (n=3, average±SD). The immunoblots below each histogram are from a representative experiment.

FIGS. 3A-3C show target-adjacent editing by CBE versus MagnEdit. FIGS. 3A and 3B are graphs plotting quantification of eGFP-positive 293T cells (Leu202 edited) post-editing (FIG. 3A) and post-enrichment by FACS (FIG. 3B) for the indicated editing reactions (n=3 technical replicate experiments, average±SD). FIG. 3C shows sequence logos summarizing MiSeq data from the same reactions as FIGS. 3A and 3B. The consensus sequence matches the ssDNA region displaced by gRNA annealing with the target cytosine. Darker coloring highlights base substitution mutations that occurred in >5% of the MiSeq reads for each reaction (numbers are nucleobase distances 5′ or 3′ of the target “C” at the zero (0) position). Top (control), SEQ ID NO:28; middle (MagnEdit), SEQ ID NO:29); bottom (CBE), SEQ ID NO:30.

FIGS. 4A-4H show the results of chromosomal DNA editing by a CBE versus MagnEdit. FIG. A is a graph plotting the percentage of eGFP-positive 293T cells (eGFP Leu202 edited with co-delivery of FANCF gRNA) post-editing and pre-enrichment by FACS for the indicated editing reactions (n=3 technical replicate experiments, average±SD). FIG. 4B shows sequence logos summarizing MiSeq data of FANCF from the same reactions as shown in FIG. 4A. The consensus sequence matches the single-stranded DNA region displaced by gRNA annealing with the target cytosine. Darker coloring highlights base substitution mutations that occurred in >5% of the MiSeq reads for each reaction (numbers are nucleobase distances 59 or 39 of the target “C”). Top (control), SEQ ID NO:31; middle (MagnEdit), SEQ ID NO:32; bottom (CBE), SEQ ID NO:33. FIG. 4C is a graph plotting the percentage of single nucleobase substitution mutations from the MagnEdit reaction shown in panel FIG. 4B. FIG. 4D is a graph plotting the editing efficiency of single nucleobase substitution mutations from the CBE reaction shown in panel FIG. 4B. FIG. 4E is a graph plotting the percentage of eGFP-positive 293T cells (eGFP Leu202 edited with co-delivery of EMX1 gRNA) post-editing and pre-enrichment by FACS for the indicated editing reactions (n=3 technical replicate experiments, average±SD). FIG. 4F contains sequence logos summarizing MiSeq data of EMX1 from the reactions used in panel FIG. 4E. The consensus sequence matches the single-stranded DNA region displaced by gRNA annealing with the target cytosine. Darker coloring highlights base substitution mutations that occurred in >5% of the MiSeq reads for each reaction (numbers are nucleobase distances 59 or 39 of the target “C”). Top (control), SEQ ID NO:34; middle (MagnEdit), SEQ ID NO:35; bottom (CBE), SEQ ID NO:36. FIG. 4G is a graph plotting the percentage of single nucleobase substitution mutations from the MagnEdit reaction shown in FIG. 4F. FIG. 4H is a graph plotting the percentage of single nucleobase substitution mutations from the CBE reaction shown in FIG. 4F.

FIGS. 5A and 5B show the results of chromosomal DNA editing in eGFP-positive versus eGFP-negative cell populations. FIG. 5A shows sequence logos summarizing MiSeq data of FANCF from eGFP-positive and eGFP-negative cell populations. For comparison, control (no gRNA) and eGFP-positive data are identical to those in FIG. 4B. Darker coloring highlights base substitution mutations that occurred in >5% of the MiSeq reads for each reaction (numbers are nucleobase distances 5′ or 3′ of the target “C”). The eGFP-negative cell populations showed similar editing trends but lower overall frequencies of both on-target and target-adjacent mutations. Sequences from top to bottom: SEQ ID NO:31, SEQ ID NO:32, SEQ ID NO:32, SEQ ID NO:33, and SEQ ID NO:37. FIG. 5B shows sequence logos summarizing MiSeq data of EMX1 from eGFP-positive and eGFP-negative cell populations. For comparison, control (no gRNA) and eGFP-positive data are identical to those in FIG. 4F. Darker coloring highlights base substitution mutations that occurred in >5% of the MiSeq reads for each reaction (numbers are nucleobase distances 5′ or 3′ of the target “C”). The eGFP-negative cell populations showed similar editing trends but lower overall frequencies of both on-target and target-adjacent mutations. Sequences from top to bottom: SEQ ID NO:34, SEQ ID NO:35, SEQ ID NO:38, SEQ ID NO:39, and SEQ ID NO:39.

### DETAILED DESCRIPTION

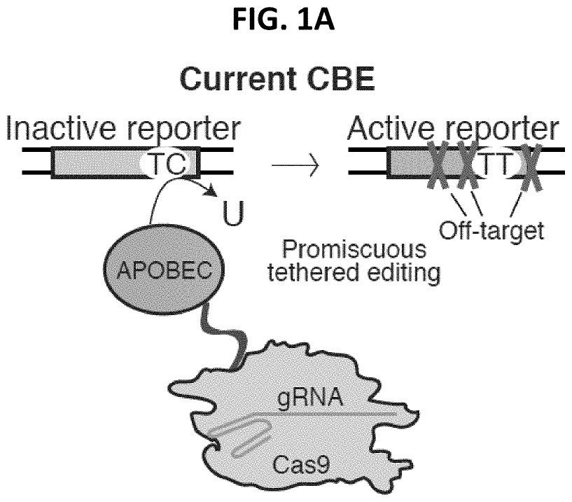

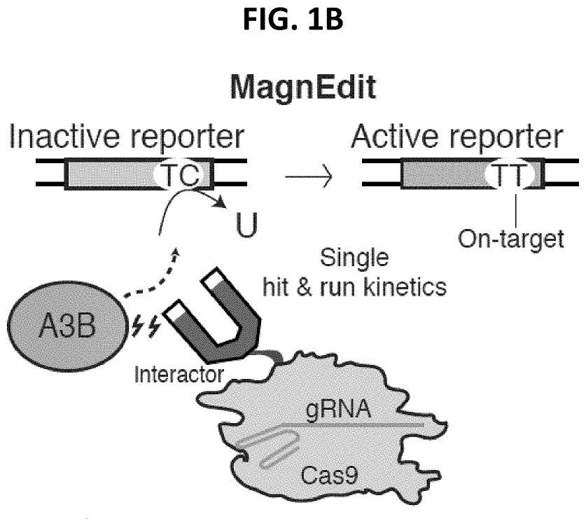

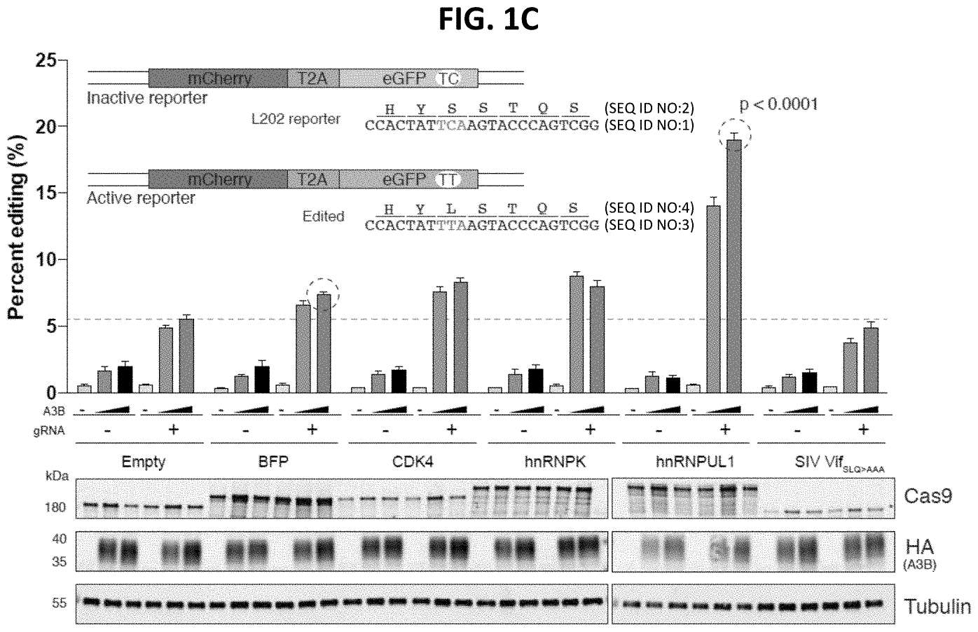

An invariant feature of previously used APOBEC-Cas9 designs is covalent fusion of the deaminase to the Cas9 complex. However, the covalent fusion may trap the tethered deaminase locally, inextricably linking both on-target and target-adjacent cytosine deamination events as illustrated in FIG. 1A. The materials and methods provided herein use non-covalent methods to “attract” a DNA cytosine deaminase to a particular genomic cytosine target. The disclosed methods can decouple the fates of on-target and target-adjacent editing events, thereby enhancing the likelihood of achieving precise single base substitution mutations. A key to implementing this non-covalent strategy is using APOBEC-interacting proteins that can bind the deaminase without blocking access to the active site. Such interacting proteins can then be tethered to a Cas9n/gRNA complex and used to “attract” a co-expressed APOBEC enzyme (e.g., an exogenous or endogenous APOBEC enzyme) to edit a particular genomic target cytosine. This novel system is referred to herein as “MagnEdit,” and is illustrated in FIG. 1B.

The materials and methods disclosed herein provide a fundamentally different approach to single base editing through the use of non-covalent interactions to “attract” a DNA cytosine deaminase to a single target cytosine. While any suitable cytosine deaminase enzyme can be used in the systems and methods provided herein, APOBEC3B (A3B) can be particularly useful in some embodiments. A3B typically is nuclear rather than shuttling or cytoplasmic like related family members (Lackey et al., J Mol Biol 419, 301-314, 2012; Lackey et al., Cell Cycle 12, 762-772, 2013; Salamango et al., J Mol Biol 430, 2695-2708, 2018; Bennett et al., Biochem Biophys Res Commun 350, 214-219, 2006; and Patenaude et al., Nat Struct Mol Biol 16, 517-527, 2009). In addition, due to active site structural constraints (Shi et al., Sci Rep 7, 17415, 2017; Wagner et al., J Chem Inf Model 59, 2264-2273, 2019; and Shi et al., Nature Struct Mol Biol 24, 131-139, 2017), A3B is less likely to elicit RNA level off-target editing events such as those documented elsewhere for BE3 and A3A CBEs (Grünewald et al., supra; and Zhou et al., supra).

Any appropriate method (e.g., proteomic, genetic, and/or directed-evolution techniques) can be used to identify APOBEC-interacting “baits” for the MagnEdit system in addition to those utilized in the Examples described herein, or to identify different interactors for the adenosine base editing systems. It is noted that proteins that interact with the non-catalytic N-terminal domain of A3B [e.g., heterogeneous nuclear ribonucleoprotein U-like (hnRNPUL1)] may be particularly effective as compared to those that bind the catalytic C-terminal domain, because they are less likely to interfere with catalytic activity. For instance, EBV BORF2 is an A3B catalytic domain interactor (Cheng et al., Nat Microbiol 4, 78-88, 2019) and, as shown in the Examples herein, it potently blocks editing in the MagnEdit system.

In some embodiments, therefore, this document provides fusion polypeptides containing an APOBEC-interacting portion and a DNA-targeting (e.g., Cas9) portion. The term “polypeptide” as used herein refers to a molecule of two or more subunit amino acids regardless of post-translational modification (e.g., phosphorylation or glycosylation). The subunits may be linked by peptide bonds or other bonds such as, for example, ester or ether bonds. The term “amino acid” refers to either natural and/or unnatural or synthetic amino acids, including D/L optical isomers.

An “isolated” or “purified” polypeptide is a polypeptide that is separated to some extent from the cellular components with which it is normally found in nature (e.g., other polypeptides, lipids, carbohydrates, and nucleic acids). A purified polypeptide can yield a single major band on a non-reducing polyacrylamide gel. A purified polypeptide can be at least about 75% pure (e.g., at least about 80%, at least about 85%, at least about 90%, at least about 95%, at least about 97%, at least about 98%, at least about 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

Nucleic acids encoding DNA-targeted APOBEC-interacting-Cas9 fusion polypeptides also are provided herein. The terms “nucleic acid” and “polynucleotide” are used interchangeably, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

As used herein, the term “isolated” in reference to a nucleic acid molecule refers to a nucleic acid that is separated from other nucleic acids that are present in a genome, e.g., a plant genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus (e.g., a pararetrovirus, a retrovirus, lentivirus, adenovirus, or herpes virus), or the genomic DNA of a prokaryote or eukaryote. In addition, an isolated nucleic acid can include a recombinant nucleic acid such as a DNA molecule that is (or is part of) a hybrid or fusion nucleic acid (e.g., a nucleic acid encoding a fusion protein as described herein). A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by any appropriate method, including, for example, chemical synthesis, polymerase chain reaction (PCR), or restriction cloning techniques. PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Recombinant nucleic acid constructs (e.g., vectors) also are provided herein. A “vector” is a replicon, such as a plasmid, phage, or cosmid, into which another DNA segment (e.g., a sequence encoding a fusion polypeptide) may be inserted so as to bring about the replication of the inserted segment. Generally, a vector is capable of replication when associated with the proper control elements. Suitable vector backbones include, for example, plasmids, viruses, artificial chromosomes, BACs, YACs, or PACs. The term “vector” includes cloning and expression vectors, as well as viral vectors and integrating vectors. An “expression vector” is a vector that includes one or more expression control sequences, and an “expression control sequence” is a DNA sequence that controls and regulates the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Takara Bio USA (Mountain View, Calif.), Stratagene (La Jolla, Calif.), Invitrogen/Life Technologies (Carlsbad, Calif.), ThermoFisher Scientific (Waltham, Mass.), and New England Biolabs (Ipswich, Mass.).

The terms “regulatory region,” “control element,” and “expression control sequence” refer to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory regions that can reside within coding sequences, such as secretory signals, Nuclear Localization Sequences (NLS) and protease cleavage sites.

As used herein, “operably linked” means incorporated into a genetic construct so that expression control sequences effectively control expression of a coding sequence of interest. A coding sequence is “operably linked” and “under the control” of expression control sequences in a cell when RNA polymerase is able to transcribe the coding sequence into RNA, which if an mRNA, then can be translated into the protein encoded by the coding sequence. Thus, a regulatory region can modulate, e.g., regulate, facilitate or drive, transcription in the plant cell, plant, or plant tissue in which it is desired to express a modified target nucleic acid.

A promoter is an expression control sequence composed of a region of a DNA molecule, typically within 1000 nucleotides upstream of the point at which transcription starts (generally near the initiation site for RNA polymerase II). Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element.

An “effective amount” of an agent (e.g., an APOBEC-interacting-Cas9 fusion polypeptide, a nucleic acid encoding such a polypeptide, or a composition containing an APOBEC-interacting-Cas9 fusion polypeptide and a gRNA directing the fusion to a specific DNA sequence) is an amount of the agent that is sufficient to elicit a desired response. For example, an effective amount of an APOBEC-interacting-Cas9 fusion polypeptide can be an amount of the polypeptide that is sufficient to induce deamination at a specific, selected target site. It is to be noted that the effective amount of an agent as provided herein can vary depending on various factors, such as, for example, the specific allele, genome, or target site to be edited, the cell or tissue being targeted, and the agent being used.

Any appropriate APOBEC-interacting polypeptide can be used in the fusion polypeptides provided herein. In some embodiments, for example, hnRNPUL1 can be particularly useful, as noted above. A representative nucleotide sequence encoding hnRNPUL1 is set forth in SEQ ID NO:8. In some cases, a fusion polypeptide provided herein can be encoded by a nucleic acid that includes a nucleotide sequence having at least about 90% identity (e.g., at least about 91%, at least about 92%, at least about 93%, at least about 94%, at least about 95%, at least about 96%, at least about 97%, at least about 98%, at least about 99%, at least about 99.5%, or at least about 99.8% identity) to the sequence set forth in SEQ ID NO:8.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\B12seq c:\seq1.txt -j c:\seq2.txt -p blastn -o c:\output.txt -q -1 -r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:8), or by an articulated length (e.g., 100 consecutive nucleotides or amino acid residues from a sequence set forth in an identified sequence), followed by multiplying the resulting value by 100. For example, an amino acid sequence that has 2500 matches when aligned with the sequence set forth in SEQ ID NO:8 is 95.6 percent identical to the sequence set forth in SEQ ID NO:8 (i.e., 2500/2614×100=95.6). It is noted that the percent sequence identity value is rounded to the nearest tenth. For example, 75.11, 75.12, 75.13, and 75.14 is rounded down to 75.1, while 75.15, 75.16, 7.17, 75.18, and 7.19 is rounded up to 7.2. It also is noted that the length value will always be an integer.

In some embodiments, the APOBEC-interacting polypeptide can be an antibody (or an antigen-binding fragment thereof) that can interact with an APOBEC enzyme. As used herein, the terms “antibody” or “antibodies” include intact molecules (e.g., polyclonal antibodies, monoclonal antibodies, humanized antibodies, or chimeric antibodies) as well as fragments thereof (e.g., single chain Fv antibody fragments, Fab fragments, and F(ab)₂ fragments) that are capable of binding to an epitopic determinant of a cytosine deaminase. An epitope is an antigenic determinant on an antigen to which the paratope of an antibody binds. Epitopic determinants typically consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). Polyclonal antibodies are heterogeneous populations of antibody molecules that are contained in the sera of the immunized animals. Monoclonal antibodies are homogeneous populations of antibodies to a particular epitope of an antigen.

Antibody fragments that can bind to a cytosine deaminase (e.g., an APOBEC) enzyme can be generated by any suitable technique. For example, F(ab′)2 fragments can be produced by pepsin digestion of an antibody molecule, and Fab fragments can be generated by reducing the disulfide bridges of F(ab′)2 fragments. Alternatively, Fab expression libraries can be constructed. See, for example, Huse et al., Science 246:1275, 1989. Once produced, antibodies or fragments thereof can be tested for recognition of a target cytosine deaminase by standard immunoassay methods, including ELISA techniques, radioimmunoassays, and Western blotting.

Antibodies having specific binding affinity for a cytosine deaminase (e.g., an APOBEC) can be produced using, for example, standard methods. See, for example, Dong et al., Nature Med 8:793-800, 2002. In general, a cytosine deaminase polypeptide can be recombinantly produced or can be purified from a biological sample, and then can be used to immunize an animal in order to induce antibody production.

The APOBEC-interacting portion of the fusion polypeptides provided herein can interact with any suitable APOBEC protein. Vertebrates encode variable numbers of APOBEC enzymes (Conticello, Genome Biol 9:229, 2008; and Harris and Dudley, Virology 479-480C:131-145, 2015), which catalyze hydrolytic deamination of cytidine or deoxycytidine in polynucleotides to uridine or deoxyuridine, respectively. All vertebrate species have activation-induced deaminase (AID), which is essential for antibody gene diversification through somatic hypermutation and class switch recombination (Di Noia and Neuberger, Annu Rev Biochem 76:1-22, 2007; and Robbiani and Nussenzweig, Annu Rev Pathol 8:79-103, 2013). Most vertebrates also have APOBEC1, which edits cytosine nucleobases in RNA and single-stranded DNA (ssDNA), and functions in regulating the transcriptome and likely also in blocking the spread of endogenous and exogenous mobile elements such as viruses (Fossat and Tam, RNA Biol 11:1233-1237, 2014; and Koito and Ikeda, Front Microbiol 4:28, 2013). The APOBEC3 subfamily of enzymes is specific to mammals, subject to extreme copy number variation, elicits strong preferences for ssDNA, and provides innate immune protection against a wide variety of DNA-based parasites, including common retrotransposons L1 and Alu, and retroviruses such as HIV-1 (Harris and Dudley, supra; Malim and Bieniasz, Cold Spring Harb Perspect Med 2:a006940, 2012; and Simon et al., Nat Immunol 16:546-553, 2015).

Human cells can produce up to seven distinct APOBEC3 enzymes, (A3A, A3B, A3C, A3D, A3F, A3G, and A3H), although most cells express subsets due to differential gene regulation (Refsland et al., Nucleic Acids Res 38:4274-4284, 2010; Koning et al., J Virol 83:9474-9485, 2009; Stenglein et al., Nat Struct Mol Biol 17:222-229, 2010; and Burns et al., Nature 494:366-370, 2013a). The local substrate preference of each APOBEC enzyme for RNA or ssDNA is an intrinsic property that has helped to elucidate biological and pathological functions for several family members. See, e.g., Di Noia and Neuberger, supra; Robbiani and Nussenzweig, supra; Harris and Dudley, supra; Malim and Bieniasz, supra; Simon et al., supra; Helleday et al., Nat Rev Genet 15:585-598, 2014; Roberts and Gordenin, Nat Rev Cancer 14:786-800, 2014; and Swanton et al., Cancer Discov 5:704-712, 2015.

The APOBEC protein can be endogenously expressed (or overexpressed) or exogenously expressed. In some embodiments, therefore, the methods provided herein can include introducing into cells an exogenous APOBEC protein that can be targeted to a particular DNA sequence by a fusion polypeptide as described herein. The APOBEC polypeptide can be untagged or tagged (e.g., with polyhistidine, a FLAG® tag, or any other suitable tag). In some cases, an APOBEC polypeptide can be tagged with one or more epitopes and/or degrons, that may be useful to further mitigate off-target effects). In some cases, an antibody that binds specifically to a tag attached to an APOBEC polypeptide can be used as the APOBEC-interacting “bait” in the fusion polypeptides provided herein.

Representative human APOBEC nucleic acid and polypeptide sequences include the A3A sequence set forth in SEQ ID NO:9 (GENBANK® accession no. NM_145699), which encodes a full length human A3A polypeptide having SEQ ID NO:10 (UniProt ID P31941), and the A3B sequence set forth in SEQ ID NO:11 (GENBANK® accession no. NM_004900), which encodes a full length human A3B polypeptide having SEQ ID NO:12 (UniProt ID Q9UH17). Other human and non-human APOBEC sequences (e.g., human APOBEC1, AID, APOBEC3C, APOBEC3D, APOBEC3F, APOBEC3G, and APOBEC3H; GENBANK® accession nos. NM_001644, NM_020661, NM_014508, NM_152426, NM_145298, NM_021822, and NM_181773, respectively) also can be used in the methods provided herein. Representative amino acid sequences for these polypeptides are provided in SEQ ID NOS:22-27, respectively.

The APOBEC polypeptides used in the methods provided herein can include the full-length amino acid sequence or a catalytic fragment of an APOBEC protein (e.g., a fragment that includes the C-terminal catalytic domain). The APOBEC polypeptide also may contain a variant APOBEC polypeptide having an amino acid sequence that is at least about 90% identical to a reference APOBEC sequence or a fragment thereof (e.g., at least about 91%, at least about 92%, at least about 93%, at least about 94%, at least about 95%, at least about 96%, at least about 97%, at least about 98%, at least about 99%, at least about 99.5%, or at least about 99.8% identical to SEQ ID NO:10, SEQ ID NO:12, or a fragment thereof). In some cases, for example, an APOBEC polypeptide can consist essentially of amino acids 13 to 199 of SEQ ID NO:10, amino acids 1 to 195 of SEQ ID NO:10, amino acids 13 to 195 of SEQ ID NO:10, or a sequence that is at least about 95% identical to such a fragment of SEQ ID NO:10. In some embodiments, the APOBEC portion can lack at least amino acids 1-12 of SEQ ID NO:10, at least amino acids 196-199 of SEQ ID NO:10, or at least amino acids 1-12 and 196-199 of SEQ ID NO:10. In some embodiments, the APOBEC portion of a fusion polypeptide as provided herein can consist essentially of amino acids 193 to 382 of SEQ ID NO:12, amino acids 193 to 378 of SEQ ID NO:12, or a sequence that is at least about 95% identical to such a fragment of SEQ ID NO:12. In some embodiments, the APOBEC portion can lack at least amino acids 1-192 of SEQ ID NO:12, or at least amino acids 1-192 and 379-382 of SEQ ID NO:12.

The CRISPR/Cas system includes components of a prokaryotic adaptive immune system that is functionally analogous to eukaryotic RNA interference, using RNA base pairing to direct DNA or RNA cleavage. The Cas9 protein functions as an endonuclease, and CRISPR RNA (crRNA) and tracer RNA (tracrRNA) sequences complex with the Cas9 enzyme and direct it to a target DNA sequence (Makarova et al., Nat Rev Microbiol 9(6):467-477, 2011). The modification of a single targeting RNA can be sufficient to alter the nucleotide target of a Cas protein. The crRNA and tracrRNA can be engineered as a single cr/tracrRNA hybrid (also referred to as a “guide RNA” or “gRNA”) to direct Cas9 cleavage activity (Jinek et al., Science, 337(6096):816-821, 2012). The CRISPR/Cas system can be used in a variety of prokaryotic and eukaryotic organisms (see, e.g., Jiang et al., Nat Biotechnol, 31(3):233-239, 2013; Dicarlo et al., Nucleic Acids Res, doi:10.1093/nar/gkt135, 2013; Cong et al., Science, 339(6121):819-823, 2013; Mali et al., Science, 339(6121):823-826, 2013; Cho et al., Nat Biotechnol, 31(3):230-232, 2013; and Hwang et al., Nat Biotechnol, 31(3):227-229, 2013).

CRISPR clusters are transcribed and processed into crRNA; the correct processing into crRNA requires a trans-encoded small tracrRNA. The combination of Cas9, crRNA, and tracrRNA can then cleave linear or circular dsDNA targets that are complementary to a spacer within the CRISPR cluster. Cas9 recognizes a short protospacer adjacent motif (PAM) in the CRISPR repeat sequences, which aids in distinguishing self from non-self. Cas9 nuclease sequences and structures are well known to those of skill in the art (see, e.g., Ferretti et al., Proc Natl Acad Sci USA 98:4658-4663, 2001; Deltcheva et al., Nature 471:602-607, 2011; and Jinek supra). Cas9 orthologs also have been described in species such as S. pyogenes and S. thermophilus.

The homology region within the crRNA sequence (the sequence that targets the crRNA to the desired DNA sequence) can be between about 10 and about 40 (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40) nucleotides in length. The tracrRNA hybridizing region within each crRNA sequence can be between about 8 and about 20 (e.g., 8, 9, 10, 11, 12, 13, 14, 15, 16, 17, 18, 19, or 20) nucleotides in length. The overall length of a crRNA sequence can be, for example, between about 20 and about 80 (e.g., 20, 25, 30, 35, 40, 45, 50, 55, 60, 65, 70, 75, or 80) nucleotides, while the overall length of a tracrRNA can be, for example, between about 10 and about 30 (e.g., 10, 12, 14, 16, 18, 20, 22, 24, 26, 28, or 30) nucleotides. The overall length of a gRNA sequence, which includes a homology region and a stem loop region that contains a crRNA/tracrRNA hybridizing region and a linker-loop sequence, can be between about 30 and about 110 (e.g., 30, 35, 40, 45, 50, 55, 60, 65, 70, 75, 80, 85, 90, 95, 100, 105, 110, 115, 120, 125, or 130) nucleotides.

In some embodiments, the Cas9 portion of the fusion polypeptides provided herein can include the non-catalytic portion of a wild type Cas9 polypeptide, or a Cas9 polypeptide containing one or more mutations (e.g., substitutions, deletions, or additions) within its amino acid sequence as compared to the amino acid sequence of a corresponding wild type Cas9 protein, where the mutant Cas9 does not have nuclease activity. In some embodiments, additional amino acids may be added to the N- and/or C-terminus. For example, Cas9 protein can be modified by the addition of a VP64 activation domain or a green fluorescent protein to the C-terminus, or by the addition of nuclear-localization signals to both the N- and C-termini (see, e.g., Mali et al. Nature Biotechnol 31:833-838, 2013; and Cong et al. Science 339:819-823). A representative Cas9 nucleic acid sequence is set forth in SEQ ID NO:13, and a representative Cas9 amino acid sequence is set forth in SEQ ID NO:14. It is to be noted that the Cas9 portion of the fusion polypeptides provided herein can be any suitable Cas9 polypeptide or related complex, with the proviso that the Cas9 polypeptide or related complex can be directed by a gRNA to form an R-loop in the DNA to be modified.

An APOBEC-interacting-Cas9 fusion polypeptide as provided herein can include the full-length amino acid sequence of a Cas9 protein, or a fragment of a Cas9 protein. Typically, the Cas9-APOBEC fusion polypeptides provided herein include a Cas9 fragment that can bind to a gRNA, but does not include a functional nuclease domain. For example, the fusion may contain a non-functional nuclease domain, or a portion of a nuclease domain that is not sufficient to confer nuclease activity, or may lack a nuclease domain altogether. Thus, in some cases, an APOBEC-interacting-Cas9 fusion polypeptide can contain a fragment of Cas9, such as a fragment including the Cas9 gRNA binding domain, or a fragment that includes both the gRNA binding domain and an inactivated version of the DNA cleavage domain. The Cas portion of an APOBEC-interacting-Cas9 fusion also may contain a variant Cas polypeptide having an amino acid sequence that is at least about 90% identical to a wild type Cas9 sequence (e.g., at least about 91%, at least about 92%, at least about 93%, at least about 94%, at least about 95%, at least about 96%, at least about 97%, at least about 98%, or at least about 99%, at least about 99.5%, or at least about 99.8% identical to a wild type Cas9 amino acid sequence).

In some embodiments, the fusion polypeptides provided herein can include a “nuclease-dead” Cas9 polypeptide that lacks nuclease activity and may or may not have nickase activity (such that it cuts one strand of a double-stranded DNA), but can bind to a preselected target sequence when complexed with crRNA and tracrRNA (or gRNA). Without being bound by a particular mechanism, the use of a DNA targeting polypeptide with nickase activity, where the nickase generates a strand-specific cut on the strand opposing the uracil to be modified, can have the subsequent effect of directing repair machinery to non-modified strand, resulting in repair of the nick so both strands are modified. For example, with respect to the Cas9 sequence of SEQ ID NO:14, a Cas9 polypeptide can be a D10A Cas9 polypeptide (or a portion thereof) that has nickase activity but not nuclease activity, or a H840A Cas9 polypeptide (or a portion thereof) that has nickase activity but not nuclease activity.

In some embodiments, a “nuclease-dead” polypeptide can be a D10A H840A Cas9 polypeptide (or a portion thereof) that has neither nickase nor nuclease activity. A Cas9 polypeptide also can be a D10A D839A H840A N863A Cas9 polypeptide in which alanine residues are substituted for the aspartic acid residues at positions 10 and 839, the histidine residue at position 840, and the asparagine residue at position 863 (with respect to SEQ ID NO:14). See, e.g., Mali et al., Nature Biotechnol, supra; Jinek et al., supra; and Qi et al., Cell 152(5):1173-83, 2013.

An exemplary reference Cas9 amino acid sequence having an inactivated nuclease domain with D10A and H840A mutations (underlined) is set forth in SEQ ID NO:15. An exemplary reference Cas9 amino acid sequence having an inactivated nuclease domain with a D10A mutation (underlined) is set forth in SEQ ID NO:16. An exemplary reference Cas9 amino acid sequence having an inactivated nuclease domain with a H840A mutation (underlined) is set forth in SEQ ID NO:17.

In some embodiments, Cas9 variants containing mutations other than D10A and H840A and lacking nuclease activity are provided herein. Such variants include, without limitation, include other amino acid substitutions at D10 and H840, or other substitutions within the Cas9 nuclease domains. In some embodiments, a Cas9 variant can have one or more amino acid additions or deletions (e.g., one, two, three, four, five, six, seven, eight, nine, 10, 10 to 20, 20 to 40, 40 to 50, or 50 to 100 additions or deletions) as compared to a reference Cas9 sequence (e.g., the sequence set forth in SEQ ID NO:14. It is noted, for example, that Cas9 has two separate nuclease domains that allow it to cut both strands of a double-stranded DNA. These are referred to as the “RuvC” and “HNH” domains. Each includes several active site metal-chelating residues. In the RuvC domain, the metal-chelating residues are D10, E762, H983, and D986, while in the HNH domain, the metal-chelating residues are D839, H840, and N863. Mutation of one or more of these residues (e.g., by substituting an alanine for the natural amino acid) may convert Cas9 into a nickase, while mutating one residue from each domain can result in a nuclease-dead and nickase-dead Cas9.

The Cas9 sequences used in the fusion polypeptides provided herein also can be based on natural or engineered Cas9 molecules from organisms such as Corynebacterium ulcerans (NCBI Refs: NC_015683.1 and NC_017317.1), C. diphtheria (NCBI Refs: NC_016782.1 and NC_016786.1), Spiroplasma syrphidicola (NCBI Ref: NC_021284.1), Prevotella intermedia (NCBI Ref: NC_017861.1), Spiroplasma taiwanense (NCBI Ref: NC_021846.1), Streptococcus iniae (NCBI Ref: NC_021314.1), Belliella baltica (NCBI Ref: NC_018010.1), Psychroflexus torquisl (NCBI Ref: NC_018721.1), Streptococcus thermophilus (NCBI Ref: YP_820832.1), Listeria innocua (NCBI Ref: NP_472073.1), Campylobacter jejuni (NCBI Ref: YP_002344900.1), Neisseria meningitidis (NCBI Ref: YP_002342100.1), and Francisella novicida. RNA-guided nucleases that have similar activity to Cas9 but are from other types of CRISPR/Cas systems, such as Acidaminococcus sp. or Lachnospiraceae bacterium ND2006 Cpf1 (see, e.g., Yamano et al., Cell 165(4):949-962, 2016; and Dong et al., Nature 532(7600):522-526, 2016) also can be used in fusion polypeptides with APOBEC-interacting polypeptides.

The domains within the APOBEC-interacting-Cas9 fusion polypeptides provided herein can be positioned in any suitable configuration. In some embodiments, for example, the APOBEC-interacting portion can be coupled to the N-terminus of the Cas9 portion, either directly or via a linker. Alternatively, the APOBEC-interacting portion can be fused to the C-terminus of the Cas9 portion, either directly or via a linker. In some cases, the APOBEC-interacting portion can be fused within an internal loop of Cas9. Suitable linkers include, without limitation, an amino acid or a plurality of amino acids (e.g., five to 50 amino acids, 10 to 20 amino acids, 15 to 25 amino acids, or 25 to 50 amino acids, such as (GGGGS)ₙ (SEQ ID NO:18), (G)n, (EAAAK)ₙ (SEQ ID NO:19), (GGS)ₙ, a SGSETPGTSESATPES (SEQ ID NO:20) motif (see, e.g., Guilinger et al., Nat Biotechnol 32(6):577-582, 2014), an (XP)ₙ motif, or a combination thereof, where n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30). Suitable linkers also include organic groups, polymers, and chemical moieties. Useful linker motifs also are described elsewhere (see, e.g., Chen et al., Adv Drug Deliv Rev 65(10):1357-1369, 2013). When included, a linker can be connected to each domain via a covalent bond, for example.

Additional components that may be present in the fusion polypeptides provided herein include, such as one or more nuclear localization sequences (NLS), cytoplasmic localization sequences, export sequences (e.g., a nuclear export sequence), or sequence tags that are useful for solubilization, purification, or detection of the fusion protein. Suitable localization signal sequences and sequences of protein tags include, without limitation, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Fusion polypeptides also can include other functional domains, such as, without limitation, a domain from the bacteriophage UGI protein that is a universal inhibitor of uracil DNA glycosylase enzymes (UNG2 in human cells; see, e.g., Di Noia and Neuberger, Nature 419(6902):43-48, 2002) that can prevent the deaminated cytosine (DNA uracil) from being repaired by cellular base excision repair (see, e.g., Komor et al. 2016, supra; and Mol et al., Cell 82:701-708, 1995).

To target an APOBEC-interacting-Cas9 fusion polypeptide to a target site (e.g., a site having a point mutation to be edited), the APOBEC-interacting-Cas9 fusion can be co-expressed with a crRNA and tracrRNA, or a gRNA, that allows for Cas9 binding and confers sequence specificity to the APOBEC-interacting-Cas9 fusion polypeptide. Suitable gRNA sequences typically include guide sequences that are complementary to a nucleotide sequence within about 50 (e.g., 25 to 50, 40 to 50, 40 to 60, or 50 to 75) nucleotides upstream or downstream of the target nucleotide to be edited. The fusion polypeptides provided herein therefore can be used for targeted DNA editing, where CRISPR RNA molecules (the crRNA and tracrRNA, or a gRNA that is a cr/tracrRNA hybrid) targeted to a particular sequence (e.g., in a genome or in an extrachromosomal plasmid) act to direct the Cas9 portion of an APOBEC-interacting-Cas9 fusion polypeptide to the target sequence while also attracting an APOBEC protein to the site, resulting in modification of a cytosine residue at the desired sequence.

Thus, this document provides methods for using systems that include CRISPR-Cas9, APOBEC-interacting, and APOBEC components to generate targeted modifications within cellular (e.g., genomic or episomal) DNA sequences. The methods can include introducing, into a cell that contains a target sequence, one or more nucleic acid molecules encoding an APOBEC-interacting-Cas9 fusion polypeptide and a CRISPR RNA (e.g., a gRNA). The cell can be a prokaryotic or eukaryotic cell, such as a bacterial cell, a yeast cell, an insect cell, a plant cell, or an animal cell (e.g., a cell from or within a human or another mammal, a fish, or a bird). In some embodiments, the methods can include transforming or transfecting a cell with (i) a first nucleic acid encoding an APOBEC-interacting-Cas9 fusion polypeptide, and (ii) a second nucleic acid encoding or containing a crRNA sequence and a tracrRNA sequence (or a gRNA sequence) targeted to a DNA sequence of interest. Such methods also can include maintaining the cell under conditions in which nucleic acids (i) and (ii) are expressed. In some cases, the methods can further include introducing into the cell an APOBEC polypeptide that can interact with the APOBEC-interacting portion of the fusion polypeptide, such that the APOBEC polypeptide is attracted to the target sequence and can generate an edit at the desired location. The fusion polypeptides provided herein can be introduced into cells via vectors encoding the polypeptides, for example, or as polypeptides per se, using any suitable technique. Appropriate methods include, without limitation, sonoporation, electroporation, lipofection, or derivatives of these or other related techniques.

After a nucleic acid within the cell is contacted with an APOBEC-interacting-Cas9 fusion polypeptide and CRISPR RNA, or after a cell is transfected or transformed with an APOBEC-interacting-Cas9 fusion and a CRISPR RNA, or with one or more nucleic acids encoding the fusion and the CRISPR RNA, any suitable method can be used to determine whether mutagenesis has occurred at the target site. In some embodiments, a phenotypic change can indicate that a change has occurred the target site. PCR-based methods also can be used to ascertain whether a target site contains a desired mutation.

When a first nucleic acid encoding an APOBEC-interacting-Cas9 fusion polypeptide and a second nucleic acid containing a crRNA and a trRNA (or a gRNA) are used, the first and second nucleic acids can be included within a single construct, or in separate constructs. Thus, while in some cases it may be most efficient to include sequences encoding the APOBEC-interacting-Cas9 polypeptide, the crRNA, and the tracrRNA in a single construct (e.g., a single vector), in other cases first nucleic acid and the second nucleic acid can be present in separate nucleic acid constructs (e.g., separate vectors). In some embodiments, the crRNA and the tracrRNA also can be in separate nucleic acid constructs (e.g., separate vectors).

Further, when an additional nucleic acid encoding an APOBEC polypeptide is used, the first nucleic acid (or first and second nucleic acids) encoding the APOBEC-interacting-Cas9 polypeptide and the CRISRP RNA and the additional nucleic acid encoding the APOBEC polypeptide can be included within a single construct, or in separate constructs. Thus, while in some cases it may be most efficient to include sequences encoding the APOBEC-interacting-Cas9 polypeptide, the crRNA and the tracrRNA (or gRNA), and the APOBEC polypeptide in a single construct (e.g., a single vector), in other cases first nucleic acid (or the first and second nucleic acids) and the additional nucleic acid can be present in separate nucleic acid constructs (e.g., separate vectors). Again, a “vector” is a replicon, such as a plasmid, phage, or cosmid, into which another DNA segment may be inserted so as to bring about the replication of the inserted segment.

The fusion polypeptides described herein, nucleic acids encoding the polypeptides, and compositions containing the polypeptides or nucleic acids, can be administered to a cell or to a subject (e.g., a human, a non-human mammal such as a non-human primate, a rodent, a sheep, a goat, a cow, a cat, a dog, a pig, or a rabbit, an amphibian, a reptile, a fish, or an insect) in order to specifically modify a targeted DNA sequence. In some cases, the targeted sequence can be selected based on its association with a particular clinical condition or disease, and the administration can be aimed at treating the clinical condition or disease. The term “treating” refer to reversal, alleviation, delaying the onset, or inhibiting the progress of the condition or disease, or one or more symptoms of the condition or disease. In some cases, administration can occur after onset of the clinical condition or disease (after one or more symptoms of the condition have developed, for example, or after the disease has been diagnosed). In some cases, however, administration may occur in the absence of symptoms, such that onset or progression of the clinical condition or disease is prevented or delayed. This may be the case when the subject is identified as being susceptible to the condition, for example, or when the subject has been previously treated for the condition and symptoms have resolved, but recurrence is possible.

In some embodiments, the methods provided herein can be used to introduce a point mutation into a nucleic acid by deaminating a target cytosine. For example, the targeted deamination of a particular cytosine may correct a genetic defect (e.g., a genetic defect is associated with a clinical condition or disease). In some embodiments, the methods provided herein can be used to introduce a deactivating point mutation into a sequence encoding a gene product associated with a clinical condition or disease (e.g., an oncogene, or a gene from a virus such as an integrated HIV-1 or a latent herpes virus in an infected cell). In some cases, for example, a deactivating mutation can create a premature stop codon in a coding sequence, resulting in the expression of a truncated gene product that may not be functional, or may lack the normal function of the full-length protein.

In some embodiments, the methods provided can be used to restore the function of a dysfunctional gene. For example, the an APOBEC-interacting-Cas9 fusion polypeptides described herein can be used in vitro or in vivo to correct a disease-associated mutation (e.g., in cell culture or in a subject). Thus, this document provides methods for treating subjects identified as having a clinical condition or disease that is associated with a point mutation. Such methods can include administering to a subject an APOBEC-interacting-Cas9 fusion polypeptide, or a nucleic acid encoding an APOBEC-interacting-Cas9 fusion polypeptide, along with a CRISPR RNA (and in some cases, an APOBEC polypeptide) in an amount effective to correct the point mutation or to introduce a deactivating mutation into the sequence associated with the disease. The disease can be, without limitation, a proliferative disease, a genetic disease, or a metabolic disease.

In some embodiments, a reporter system can be used to detect activity of the fusion proteins described herein. See, for example, the luciferase-based assay described in US 2016/0304846, in which deaminase activity leads to expression of luciferase. US 2016/0304846 also describes a reporter system utilizing a reporter gene that has a deactivated start codon. In this reporter system, successful deamination of the target permits translation of the reporter gene. The Examples herein also disclose the use of a dual mCherry-T2A-eGFP reporter, which is further described in U.S. Publication No. 2019/0017055.

It is to be noted that, while the examples provided herein relate to APOBEC-interacting-Cas9 fusions that an interact with APOBEC polypeptides, the use of DNA-targeting molecules other than CRISPR-Cas is contemplated. Thus, for example, a modified APOBEC polypeptide can be coupled to a DNA-targeting domain from a polypeptide such as a meganuclease (e.g., a wild type or variant protein of the homing endonuclease family, such as those belonging to the dodecapeptide family (LAGLIDADG; SEQ ID NO:21), a transcription activator-like (TAL) effector protein, or a zinc-finger (ZF) protein. Such proteins and their characteristics, function, and use are described elsewhere. See, e.g., WO 2004/067736/Porteus, Nature 459:337-338, 2009; Porteus and Baltimore, Science 300:763, 2003; Bogdanove et al., Curr Opin Plant Biol 13:394-401, 2010; and Boch et al., Science 326(5959):1509-1512, 2009.

The invention will be further described in the following examples, which do not limit the scope of the invention described in the claims.

### EXAMPLES

### Example 1—Materials and Methods

Cell lines. 293T and 293T-Leu202 cells were cultured in RPMI 1640 supplemented with 10% fetal bovine serum (FBS) and penicillin-streptomycin. A chromosomal 293T-Leu202 reporter line was constructed using viral transduction followed by hygromycin selection (detailed below).

Constructs. The rat APOBEC1-Cas9n-UGI-NLS construct (BE3) was provided by David Liu (Komor et al. 2016, supra). Uracil DNA glycosylase inhibitor (UGI) is an 83-residue protein from Bacillus subtilis bacteriophage PBS1 that very effectively blocks human uracil DNA glycosylase activity, and its inclusion in the construct can block base-excision repair and thus boost editing efficiency. Interactor cDNA sequences were cloned into the BE3 vector in place of APOBEC1 using standard PCR subcloning techniques. blue fluorescent protein (BFP) sequence, GENBANK® accession number MK178577.1 (SEQ ID NO:5); cyclin dependent kinase 4 (CDK4) sequence, GENBANK® accession number NM_000075.4 (SEQ ID NO:6); heterogeneous nuclear ribonucleoprotein K (hnRNPK) sequence, GENBANK® accession number NM_031263.4 (SEQ ID NO:7); and hnRNPUL1 sequence, GENBANK® accession number EU831487.1 (SEQ ID NO:8). Simian immunodeficiency virus (SIV)-Vif was subcloned from a construct described elsewhere (Land et al., Oncotarget 6, 39969-39979, 2015; and Wang et al., J Virol 92, pii: e00447, 2018). Leu202 gRNA, NS gRNA, empty-Cas9n-UGI-NLS and Leu202 reporter (pLenti-CMV-mCherry-T2A-eGFP) also are described elsewhere (St. Martin et al. 2019, supra), as are pcDNA3.1-3×HA, A3Bi-3×HA and A3Biv54D-3×HA (Lackey et al., supra). A3B_{chim22-32}-3×HA was subcloned from a construct described elsewhere (Salamango et al., J Mol Biol 430, 2695-2708, 2018). BORF2-3×Flag also is described elsewhere (Chen et al., Nature Microbiol 4, 78-88, 2019).

Episomal base editing experiments. Semi-confluent 293T cells in a 6-well plate format were transfected with 200 ng gRNA, 400 ng reporter, 600 ng Cas9n-UGI-NLS, and either 600 ng pcDNA3.1-3×HA, 300 ng pcDNA3.1-3×HA and 300 ng A3B-3×HA or 600 ng A3B-3×HA [25 minutes at RT with a 3:1 ratio of TransIT LT1 (Mirus) and 250 μl of serum-free RPMI 1640 (Hyclone)]. Cells were harvested after 72 hours of incubation for editing quantification by flow cytometry.

Chromosomal base editing experiments. Semi-confluent 10 cm plates of 293T cells were transfected with 8 μg of an HIV-1 Gag-Pol packaging plasmid, 1.5 μg of a VSV-G expression plasmid, and 3 μg of pLenti-CMV-mCherry-T2A-eGFP_{Leu202}-IRES-Hygro. Viruses were harvested 48 hours post-transfection and used to transduce target cells. 48 hours post-transduction, cells were selected using 250 μg/ml Hygromycin. Transduced, mCherry-positive cells were transfected with 600 ng Cas9n-UGI editor, 200 ng of Leu202 or NS-gRNA and either 600 ng pcDNA3.1-3×HA, 300 ng pcDNA3.1-3×HA and 300 ng A3B-3×HA or 600 ng A3B-3×HA. Cells were harvested 72 hours post-transfection, and editing was quantified by flow cytometry (fraction of eGFP and mCherry double-positive cells in the total mCherry-positive population).

MiSeq. eGFP target sequences were amplified using Phusion high-fidelity DNA polymerase (NEB) and primers described elsewhere (St. Martin et al. 2019, supra). To add diversity to the sequence library, zero, one, or two extra cytosine bases were added to forward and reverse primers for each amplicon. Barcodes were added to generate full-length Illumina amplicons. Samples were analyzed using Illumina MiSeq 2×75-nucleotide paired-end reads (University of Minnesota Genomics Center). Reads were paired using FLASh (Magoc̆, T. & Salzberg, Bioinformatics 27, 2957-2963, 2011). Data processing was performed using a locally installed FASTX-Toolkit. Fastx-clipper was used to trim the 3′ constant adapter region from sequences, and a stand-alone script was used to trim 5′ constant regions. Trimmed sequences were then filtered for high-quality reads using the Fastx-quality filter. Sequences with a Phred quality score less than 30 (99.9% base calling accuracy) at any position were eliminated. Preprocessed sequences were then further analyzed using the FASTAptamer toolkit (Alam et al., Mol Ther Nucl Acids 4, e230, 2015). FASTAptamer-Count was used to determine the number of times each sequence was sampled from the population. Each sequence was then ranked and sorted based on overall abundance, normalized to the total number of reads in each population, and directed into FASTAptamer-Enrich. FASTAptamer-Enrich calculates the fold enrichment ratios from a starting population to a selected population by using the normalized reads-per-million (RPM) values for each sequence. Sequences at abundances lower than 5 RPM in the A3-editosome samples were discarded. For reporter and A3-editosome comparisons, sequences that appeared only in the A3-containing samples (with an RPM value over 5), or sequences that occurred at a frequency below 5 RPM in the no-gRNA controls were included for analysis.

Immunoblots. 1×10⁶ cells were lysed directly into 2.5× Laemmli sample buffer, separated by 4-20% SDS-PAGE, and transferred to PVDF-FL membranes (Millipore). Membranes were blocked in 5% milk in PBS and incubated with primary antibody diluted in 5% milk in PBS supplemented with 0.1% Tween20. Secondary antibodies were diluted in 5% milk in PBS supplemented with 0.1% Tween20 and 0.01% SDS. Membranes were imaged with a LI-COR Odyssey instrument. Primary antibodies used in these experiments were rabbit anti-Cas9 (Abcam ab189380), mouse anti-Tubulin (Sigma T5168), rabbit anti-HA (Cell Signaling 3724S) and mouse anti-Flag (Sigma F1804). Secondary antibodies used were goat anti-rabbit IRdye 800CW (Licor 827-08365) and goat anti-mouse Alexa Fluor 680 (Molecular Probes A-21057).

### Example 2—Episomal MagnEdit Reporter Editing

In initial experiments, several A3B-interacting proteins—SIV Vif (Land et al., Oncotarget 6, 39969-39979, 2015), hnRNPK (Zhang et al., Cell Microbiol 10, 112-121, 2008), and CDK4 (McCann et al., J Mol Biol 419, 301-314, 2012), and hnRNPUL1 (Gabler et al., J Virol 72(10):7960-7971, 1998)—were fused to the N-terminal end of Cas9n, and studies were conducted to determine whether these complexes were able to recruit A3B to edit an episomal eGFP reporter (St. Martin et al. 2019, supra) in 293T cells, resulting in conversion of TC to TT (FIG. 1B) in the eGFP gRNA target sequence (FIG. 1C, inset). Due to simultaneous overexpression of reaction components following co-transfection, including A3B, a low level of eGFP-positive cells (˜1-2%) was observed in the absence of a gRNA and a candidate interacting protein (reactions represented by “gRNA-” in FIG. 1C). Interestingly, addition of an eGFP Leu202-targeting gRNA (again without an interactor) enabled higher levels of eGFP editing by A3B (˜5-7%; “Empty” Cas9n plus gRNA reaction in FIG. 1C). Most MagnEdit complexes failed to stimulate editing beyond these background levels or those caused by a non-interacting BFP-Cas9n control (FIG. 1C). SIV Vif (SLQ-AAA)-Cas9n even yielded lower overall frequencies of background editing, likely due to poorer expression relative to other MagnEdit constructs (the SLQ-AAA was necessary to prevent Vif from binding ELOC and triggering A3B degradation; Land et al., supra). However, one MagnEdit construct, hnRNPUL1-Cas9n, was clearly capable of recruiting A3B in a dose-dependent manner to catalyze editing and activation of the eGFP reporter (FIG. 1C). Editing frequencies due to hnRNPUL1-Cas9n were at least 2-fold higher than the BFP-Cas9n/gRNA-induced background in these transient transfection experiments (p<0.0001 by unpaired student's t-test).

### Example 3—Genomic MagnEdit Reporter Editing

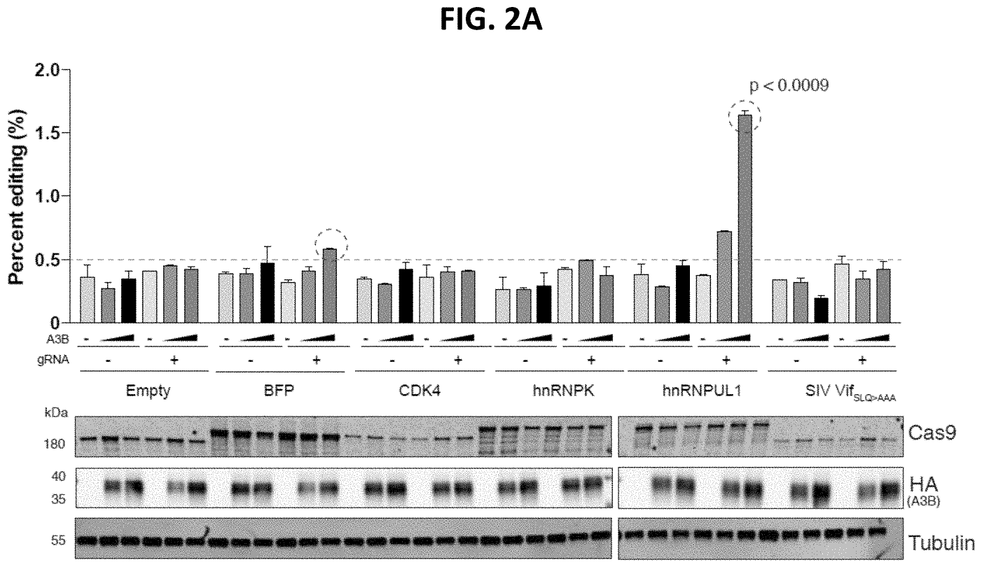

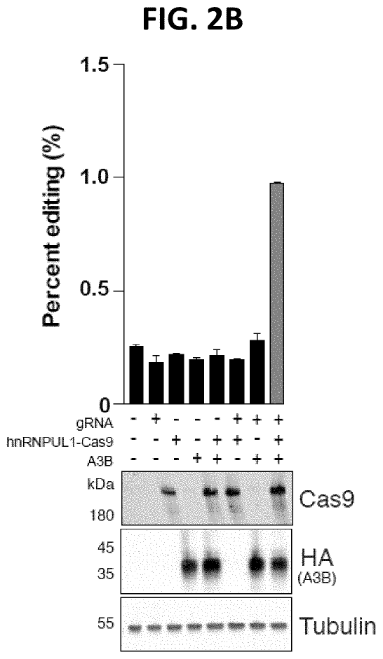

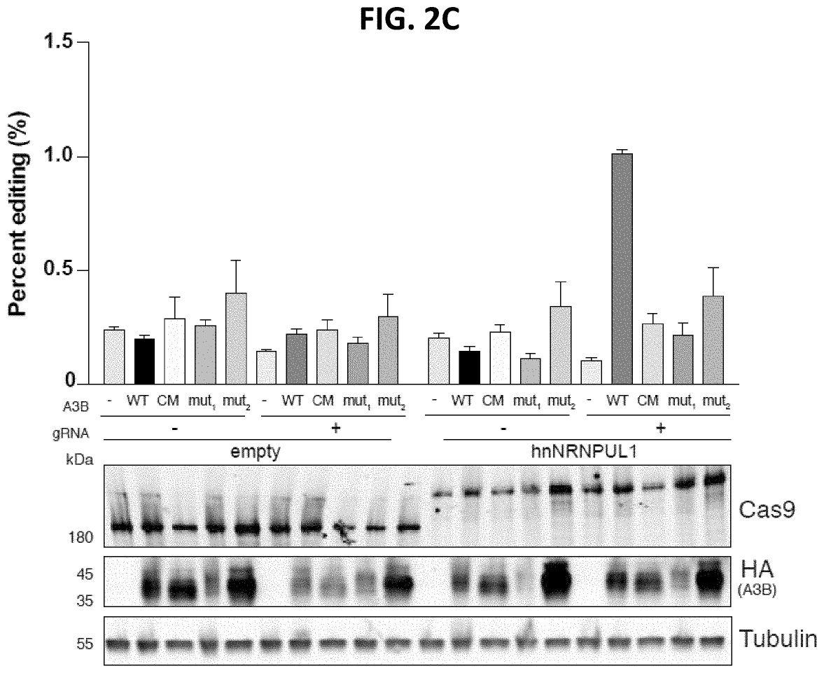

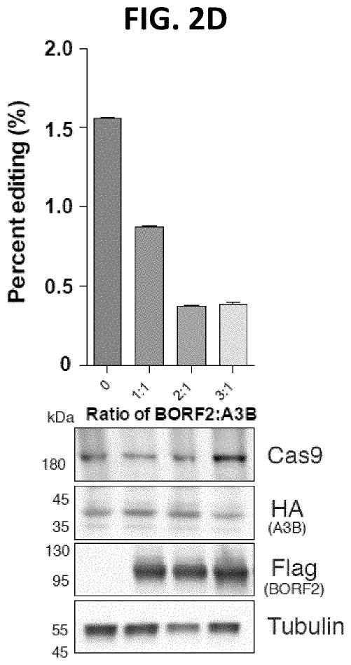

Next, chromosomal DNA editing by MagnEdit was analyzed. The eGFP Leu202 reporter was integrated into the genome of 293T cells by low MOI lentiviral transduction, followed by hygromycin selection to ensure that every cell had one editing target (uniform mCherry-positive population confirmed by flow cytometry). This pool was then transfected, as above, with the panel of A3B interactor-Cas9n complexes with or without the Leu202 targeting gRNA in the presence or absence of exogenous A3B. Also as above, empty-Cas9n and BFP-Cas9n were used as negative controls. In these studies, most MagnEdit again complexes showed activity that was not above background levels. Flow cytometry noise was the likely source of these low background levels of eGFP positivity, because no difference was observed with/without the eGFP Leu202 targeting gRNA or different amounts of A3B. In agreement with the episomal editing data, however, hnRNPUL1 MagnEdit complexes yielded a dose-dependent increase in A3B editing (quantification and representative immunoblots in FIG. 2A; p<0.0009 by unpaired student's t-test). As expected, all components of the MagnEdit reaction (the hnRNPUL1-Cas9n complex, Leu202 gRNA, and A3B-HA) were required for chromosomal DNA editing (FIG. 2B).

### Example 4—Nuclear Import Activity is Required for Genomic MagnEdit Editing

To further investigate the mechanistic requirements for MagnEdit, studies were conducted to determine whether the nuclear import activity of A3B was required. A3B is the only constitutively expressed nuclear human APOBEC family member (Lackey et al., supra; Lackey et al. 2013, supra; and Salamango et al., supra), and nuclear localization was predicted to be essential for MagnEdit. Studies described elsewhere have combined to delineate a non-canonical nuclear import mechanism involving multiple A3B surface residues in two distinct patches (Salamango et al., supra). Indeed, two previously characterized import-defective mutants, Va154Asp (Lackey et al. 2012, supra) and chim 22-32 (Salamango et al., supra), were not capable of editing the chromosomal eGFP Leu202 reporter (FIG. 2C). The amino acid substitutions within Va154Asp and chim 22-32 are localized to the A3B N-terminal regulatory domain, and their editing phenotypes were indistinguishable from that of a C-terminal domain catalytic mutant (CM in FIG. 2C). Additionally, the chromosomal DNA editing reaction was suppressed in a dose-dependent manner by BORF2, an A3B antagonist encoded by Epstein-Barr virus (Cheng et al., supra) (FIG. 2D).

### Example 5—MagnEdit Reduces Off-Target Editing

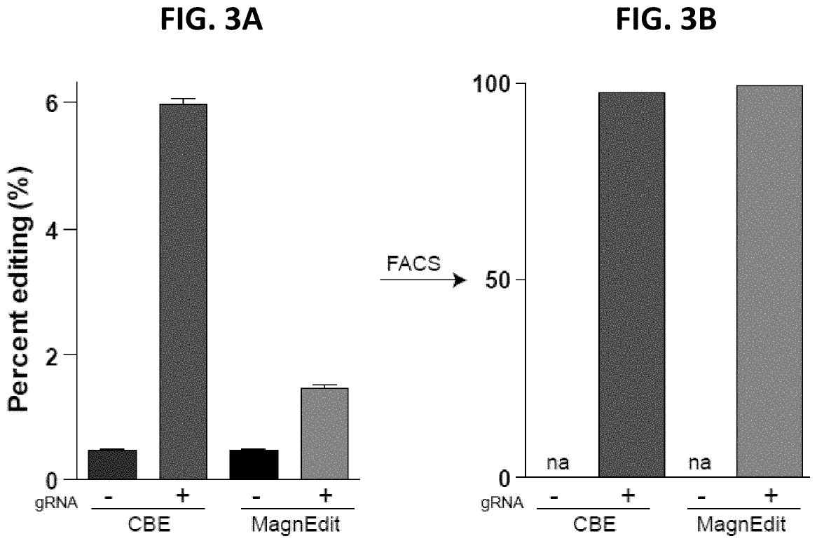

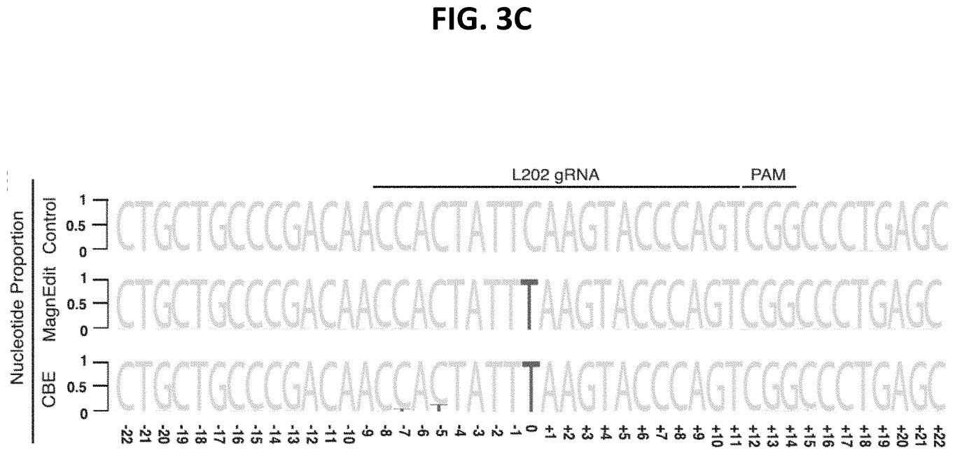

In further studies, DNA sequencing was used to compare the ratios of on-target and target-adjacent editing by a current CBE (A3B-Cas9n) (St. Martin et al. 2019, supra) and the MagnEdit complex described herein (A3B plus hnRNPUL1-Cas9n). A3B-Cas9n was used for these comparisons because its catalytic domain is less promiscuous than BE3 (St. Martin et al. 2019, supra), and it provides an isogenic comparison for covalent versus non-covalent editing reactions catalyzed by A3B. As above, chromosomal DNA editing was performed by transfecting Cherry-positive 293T pools with the eGFP Leu202 gRNA expression vector and plasmids encoding either A3B-Cas9n or hnRNPUL1-Cas9n with a separate vector for A3B. FACS was used 72 hours post-transfection to isolate eGFP-positive positive pools for target recovery and deep sequencing. As indicated by bright eGFP-positive signals in each editing reaction 72 hours post-transfection, both editing technologies activated the reporter, with the A3B CBE appearing only 4-fold more efficient (6.1% for A3B-Cas9n vs. 1.5% for A3B plus hnRNPUL1-Cas9n) (FIG. 3A). In each instance, FACS resulted in enrichment of similar numbers of eGFP-positive cells for deep sequencing (98% for A3B-Cas9n and 99% for A3B plus hnRNPUL1-Cas9n) (FIG. 3B).

As negative controls, parallel reactions without gRNAs were directly converted to genomic DNA for deep sequencing, and no target cytosine mutations were observed. In contrast, as anticipated above and from studies described elsewhere (St. Martin 2019, supra), the inclusion of a gRNA enabled both technologies to restore functionality to eGFP codon 202 [TCA (Ser) to TTA (Leu); represented by a black T and normalized to 1 for comparisons in FIG. 3C]. However, target-adjacent editing frequencies were clearly different for these two different base editing technologies. The covalently tethered A3B-Cas9n CBE caused high frequencies of target-adjacent editing within the R-loop created by gRNA-interacting region (27% at the -5 position and 16% at the -7 position in FIG. 3C). In contract, the hnRNPUL1-Cas9n MagnEdit system showed much lower target-adjacent editing within the gRNA-interacting region (0.9% at the -5 position and 3.6% at the -7 position in FIG. 3C). Thus, these results combined to demonstrate that MagnEdit is capable of yielding high frequencies of on-target editing with significantly lower frequencies of target-adjacent editing events.

### Example 6—Chromosomal DNA Editing by CBE Versus MagnEdit

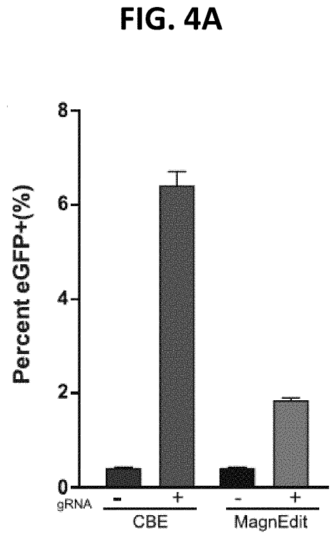

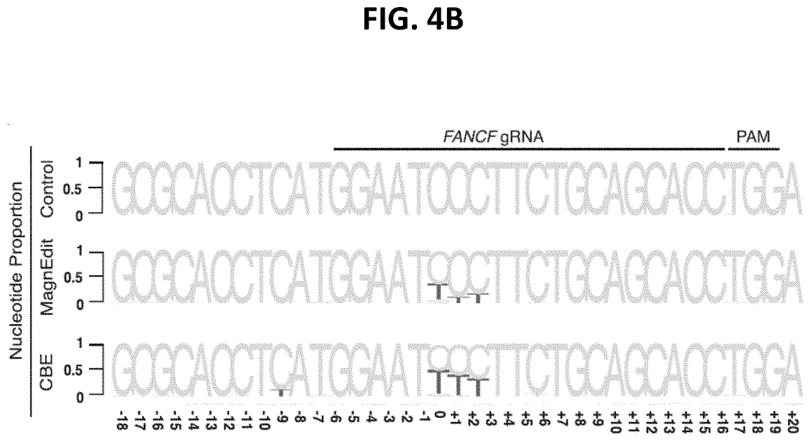

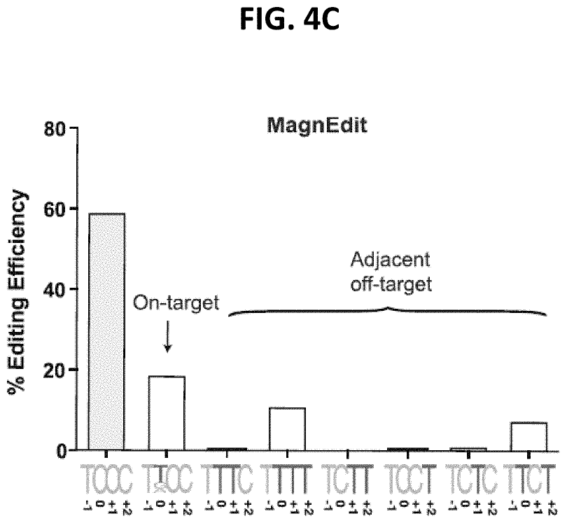

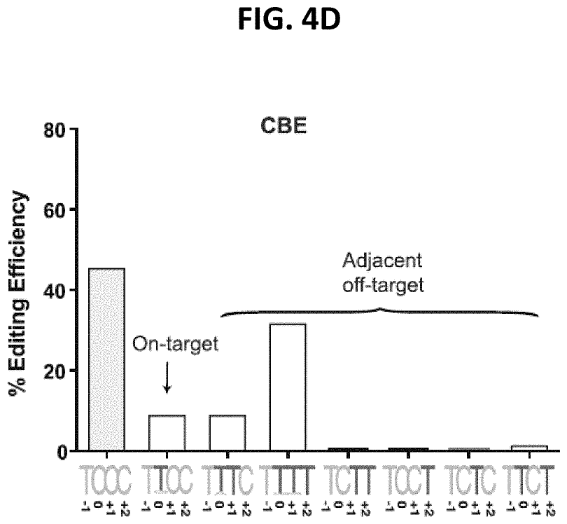

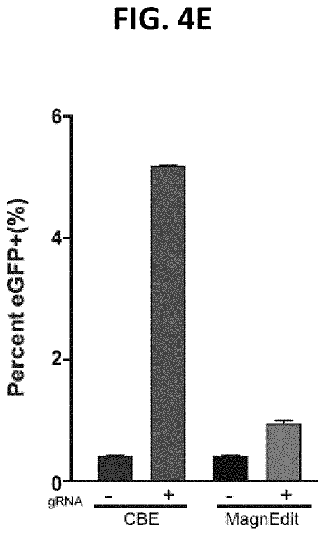

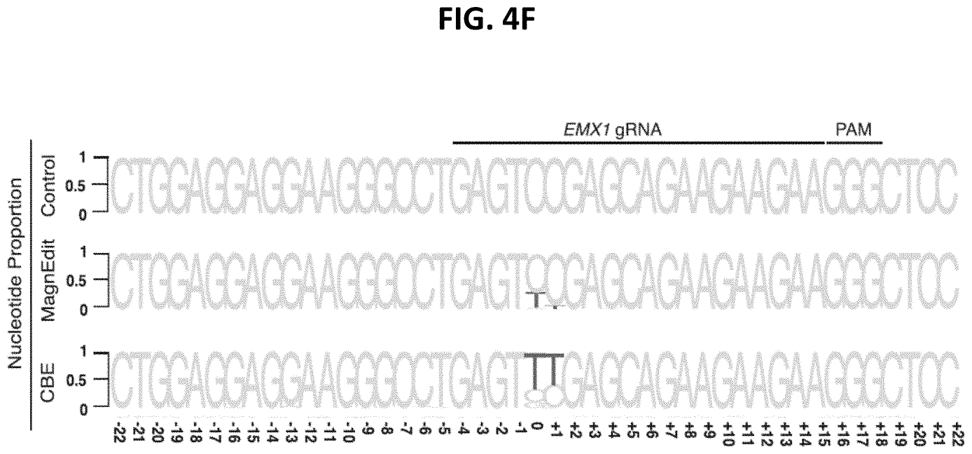

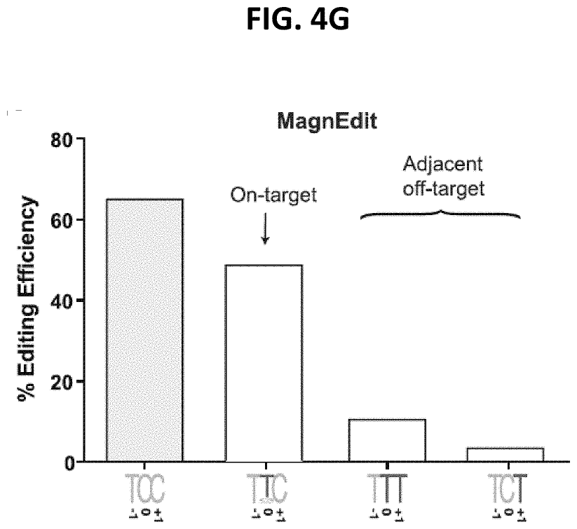

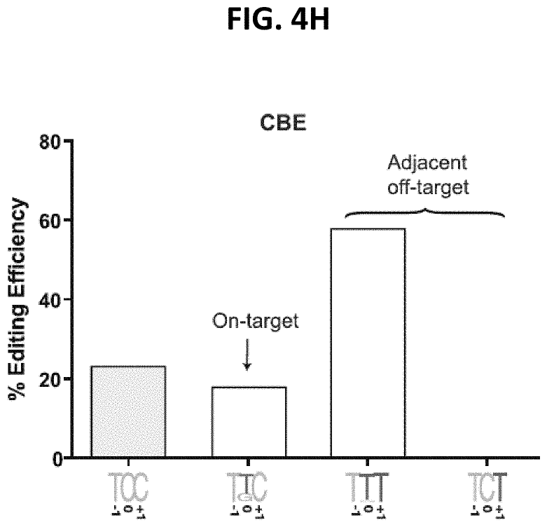

To further investigate the accuracy of the MagnEdit system, the ratios of on-target and target-adjacent editing were compared by a current CBE (A3BCas9n) (St. Martin et al. 2019, supra) and the MagnEdit complex described herein (A3B plus hnRNPUL1-Cas9n) at two genomic loci, FANCF and EMX1 (Komor et al. 2016, supra). As above, chromosomal DNA editing was performed by transfecting Cherry-positive 293T pools with gRNAs targeting both the eGFP Leu202 reporter and FANCF or EMX1 and plasmids encoding either A3B-Cas9n or hnRNPUL1-Cas9n with a separate vector for A3B. FACS was used 72 hours post-transfection to isolate eGFP-positive pools for target DNA recovery and deep sequencing. Similar to the results shown in FIGS. 3A and 3B, both editing technologies activated the eGFP reporter with, again, the A3B CBE appearing about fourfold more efficient (FIGS. 4A and 4E).

As negative controls, parallel reactions without gRNAs were directly converted to genomic DNA for deep sequencing, and no target cytosine mutations were observed in FANCF or EMX1 (control reactions in FIGS. 4B and 4F). Upon inclusion of appropriate gRNAs targeting these genes, however, clear differences in accuracy were observed between these two different base editing technologies. Similar to FANCF editing by BE3 (Komor et al. 2016, supra), the covalently tethered A3B-Cas9n CBE caused high frequencies of target-adjacent editing within the R-loop created by gRNA binding (42% at the +1 position and 35% at the +2 position in FIG. 4B). It also caused significant off-target editing at the −9 position, which is just upstream of the gRNA-binding region (13.9% in FIG. 4B). In contrast, the hnRNPUL1-Cas9n MagnEdit system showed significantly lower target-adjacent editing within the gRNA-binding region and no detectable editing outside of the gRNA-binding region (13% at the +1 position, 20% at the +2 position, and 0.5% at the -9 position in FIG. 4B). Although target-adjacent editing was higher in FANCF than in the eGFP L202 reporter, this was likely due to the trinucleotide context of FANCF being “TCC” rather than “TCA” (that is, TCC is a suboptimal context for A3B as shown by biochemical and structural studies (Shi et al., Nature Struct Mol Biol 24, 131-139, 2017)). Nevertheless, upon consideration of all possible editing permutations within the gRNA-binding region (on-target and target-adjacent events), the hnRNPUL1-Cas9n MagnEdit system showed a twofold increase in on-target editing in comparison to the covalently tethered A3B-Cas9n CBE (19% versus 9% in FIGS. 4C and 4D, respectively). The hnRNPUL1-Cas9n MagnEdit system yielded correspondingly fewer target-adjacent editing events than the A3BCas9n CBE system (21.8% versus 45.5% in FIGS. 4C and 4D, respectively).

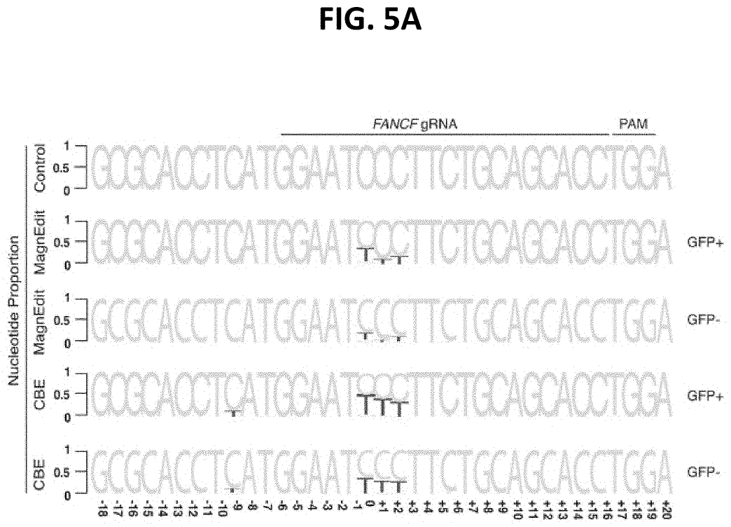

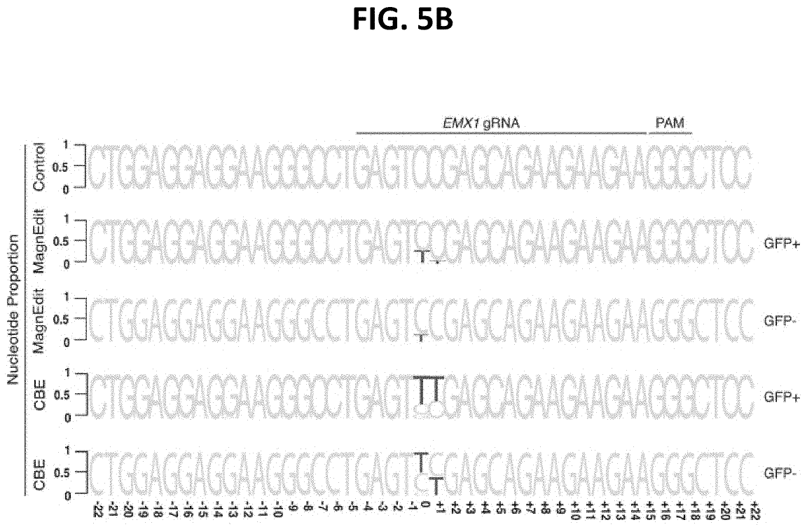

Similar trends were evident for the chromosomal EMX1 locus. The covalently tethered A3B-Cas9n CBE caused high frequencies of target-adjacent editing within the R-loop created by the gRNA binding (58.5% at the +1 position in FIG. 4F). In contrast, the hnRNPUL1-Cas9n MagnEdit system showed more than threefold lower target-adjacent editing within the gRNA-binding region (15.0% at the +1 position in FIG. 4F). Again, this genomic target has a trinucleotide context of “TCC” rather than “TCA,” so editing results were broken down into trinucleotide contexts for further consideration. The hnRNPUL1-Cas9n MagnEdit system specifically edited the target “C,” whereas the covalently tethered A3B-Cas9n CBE was less specific (49% versus 18.2% on-target editing, respectively, FIGS. 4G and 411). In combination, these results demonstrated that the MagnEdit system yields higher frequencies of on-target editing, along with significantly lower frequencies of target-adjacent editing events. In addition, higher FANCF and EMX1 on-target editing frequencies and similar adjacent off-target trends were evident for MagnEdit versus the covalently tethered A3B-Cas9n CBE in eGFP-negative pools (FIGS. 5A and 5B). These additional results from sequencing the “dark” population suggested that on-target chromosomal editing events may far exceed those that yielded functional correction of the eGFP Leu202 reporter.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A fusion polypeptide comprising:
(a) an apolipoprotein B mRNA editing enzyme, catalytic polypeptide-like-(APOBEC-) interacting polypeptide, and
(b) a Cas9 polypeptide.

2. The fusion polypeptide of claim 1, wherein the APOBEC-interacting polypeptide is N-terminal of the Cas9 polypeptide.

3. The fusion polypeptide of claim 1, wherein the APOBEC-interacting polypeptide is a heterogeneous nuclear ribonucleoprotein U-like (hnRNPUL1) polypeptide.

4. The fusion polypeptide of claim 3, wherein the hnRNPUL1 polypeptide is encoded by a nucleic acid sequence comprising the sequence set forth in SEQ ID NO:8, or a sequence having at least about 90% identity to SEQ ID NO:8.

5. The fusion polypeptide of claim 1, wherein the APOBEC-interacting polypeptide is an antibody or an antigen binding portion thereof.

6. The fusion polypeptide of claim 5, wherein the antibody or antigen-binding portion thereof is a single chain antibody or an antigen binding portion thereof.

7. The fusion polypeptide of claim 1, wherein the Cas9 polypeptide is encoded by a nucleic acid sequence comprising the sequence set forth in SEQ ID NO:13, or a sequence having at least about 90% identity to SEQ ID NO:13, with the proviso that in the encoded Cas9 polypeptide, that the amino acid at the position corresponding to position 10 of SEQ ID NO:14 is A1a, the amino acid at the position corresponding to position 840 of SEQ ID NO:14 is A1a, or the amino acids at the positions corresponding to positions 10 and 840 of SEQ ID NO:14 are A1a.

8. A nucleic acid molecule comprising a nucleotide sequence encoding the fusion polypeptide of claim 1.

9. The nucleic acid of claim 8, wherein the nucleic acid molecule is an expression vector.

10. A host cell comprising the nucleic acid molecule of claim 9.

11. A method for inducing DNA base editing at a specific DNA target in a cell, comprising introducing into the cell:
(a) a first nucleic acid encoding a fusion polypeptide, wherein the first nucleic acid comprises (i) a sequence encoding an APOBEC-interacting polypeptide, and (ii) a sequence encoding a Cas9 polypeptide;
(b) a guide RNA (gRNA) targeted to the specific DNA target.

12. The method of claim 11, further comprising introducing into the cell:
(c) a nucleic acid encoding an APOBEC polypeptide.

13. The method of claim 12, wherein the APOBEC polypeptide is an APOBEC3B polypeptide.

14. The method of claim 11, wherein the sequence encoding the APOBEC-interacting polypeptide is 5′ of the sequence encoding the Cas9 nickase.

15. The method of claim 11, wherein the APOBEC-interacting polypeptide is a hnRNPUL1 polypeptide.

16. The method of claim 15, wherein the hnRNPUL1 polypeptide is encoded by a nucleic acid sequence comprising the sequence set forth in SEQ ID NO:8, or a sequence having at least about 90% identity to SEQ ID NO:8.

17. The method of claim 11, wherein the Cas9 polypeptide is encoded by a nucleic acid sequence comprising the sequence set forth in SEQ ID NO:13, or a sequence having at least about 90% identity to SEQ ID NO:13, with the proviso that in the encoded Cas9 polypeptide, the amino acid at the position corresponding to position 10 of SEQ ID NO:14 is A1a, the amino acid at the position corresponding to position 840 of SEQ ID NO:14 is A1a, or the amino acids at the positions corresponding to positions 10 and 840 of SEQ ID NO:14 are A1a.

18. The method of claim 11, wherein the cell is a primary human cell.

19. The method of claim 11, wherein the cell is a stem cell, a lymphocyte, or a hepatocyte.
